(19) United States
(12) Patent Application Publication
(10) Pub. No.: US 2024/0207356 A1
(43) Pub. Date: Jun. 27, 2024

(54) COMPOSITION AND METHOD FOR WOUND HEALING AND REPAIR OF DAMAGED NERVES

(71) Applicant: PHARMAZZ, INC., Willowbrook, IL (US)

(72) Inventor: Kartike Gulati, Willowbrook, IL (US)

(73) Assignee: PHARMAZZ, INC., Willowbrook, IL (US)

(21) Appl. No.: 18/146,396

(22) Filed: Dec. 26, 2022

(51) Int. Cl. A61K 38/10; A61K 45/06; A61P 17/02; A61P 25/00

(52) U.S. Cl. CPC A61K 38/10; A61K 45/06; A61P 17/02; A61P 25/00

(57) ABSTRACT

The aspects of the disclosed embodiments relate to compositions and methods for treating wounds and or promoting wound healing and mammalian nerve regeneration of a damaged nerve. In addition, the invention relates to the application of the therapeutic amount of an endothelin-B receptor agonist. Specifically, this application is directed to a method of using IRL-1620 (sovateltide) for treating dermal or cutaneous wounds and damaged cranial or peripheral nerves. The composition may include endothelin-B receptor agonists, which may include peptides such as sovateltide (IRL-1620). Delivery systems, including gels, sponges, gauzes, and meshes incorporating the agent for topical application, are also described.

### FIELD

The present disclosure is related to methods and compositions for promoting the rate and improving the quality of wound healing, including neurovascular and muscle tissue regeneration in an individual in need thereof. The method comprises administering an endothelin B (ETB) receptor agonist with one or more bioactive factors or stem cells.

### BACKGROUND

The healing of wounds is a complex process involving many stages. These include; 1) coagulation, which begins immediately after injury; 2) inflammation, which begins a few minutes later; 3) a migratory and proliferative process (granulation stage), which begins within hours to days; and 4) a remodeling process with subsequent development of full strength skin [1-3]. Coagulation controls hemostasis and initiates healing by releasing various growth factors and cytokines from degranulated platelets. During the inflammation phase, platelet aggregation and clotting form a matrix that traps plasma proteins and blood cells to induce the influx of various types of cells. Neutrophils are the first cells to arrive and function to phagocytize contaminating bacteria, digest the fibrin clot, release mediators to attract macrophages, and activate fibroblasts and keratinocytes [3]. Macrophages digest pathogens, debride the wound, and secrete cytokines/growth factors (e.g. interleukin-1 (I-1), epidermal growth factor (EGF), vascular endothelial growth factor (VEGF), transforming growth factor-β (TGF-β), and basic fibroblast growth factor (bFGF)) that stimulate fibroblasts and endothelial cells. Overall, the inflammatory stage is important to guard against infection and promote the migratory and proliferative stages of wound healing.

There are two types of wounds, acute and chronic. An acute wound takes 4-6 weeks to heal completely. When the healing process is prolonged for more than 6 weeks without resolution, it leads to a chronic wound. Chronic wounds, such as venous, diabetic, or pressure ulcers, represent one of the world's most significant unmet medical needs today and are a major complication of diabetes, resulting in significant morbidity, lost productivity, and healthcare expenditures. Diabetic foot ulceration is a significant cause of morbidity and is the most common reason for hospital admission in diabetic patients. Approximately 15% of diabetic patients will develop chronic ulcers during their lifetimes. Of those who require lower-limb amputation, 70-90% will be preceded by foot ulceration [4].

Diabetic wounds show elevated levels of matrix metalloproteinases (MMPs), increased proteolytic degradation of ECM components, and inactivation of growth factors that culminate in a corrupt ECM that cannot support healing [5, 6]. Abnormal nitric oxide (NO) production also contributes to the pathogenesis of impaired healing. Cells such as keratinocytes, fibroblasts, and macrophages display both dysfunctional expression and responses to many growth factors and cytokines. Thus, these wounds typically are non-responsive to most treatments. For these reasons, it may be most advantageous to intervene with aggressive strategies that could restore the corrupt extracellular microenvironment in a diabetic wound in a multifaceted manner closely approximating the components of the normal ECM. The regulatory pathways consist of complex networks making it difficult to design compensatory adjustments required for wound repair. Therefore, aggressive healing strategies that replace the missing or dysfunctional extracellular matrix (ECM) components may be most advantageous.

### BACKGROUND OF PRIOR ART

During the past decade, vascular endothelial growth factor (VEGF) has been widely investigated and reported to have pleiotropic functions in the central nervous system (CNS) and its supporting physiological environment. VEGF is involved in well-known functions such as angiogenesis, accentuation of vessel permeability, and glial proliferation and more recently acknowledged functions such as neuroprotection and even neurogenesis. Most recently, the neurogenesis function has attracted much attention, and several research groups have taken up the challenge of elucidating this activity. In keeping with this trend, our knowledge of VEGF receptors has increased, and certain suggestions concerning the mechanisms of neuro protection have come to light in the course of the ongoing work, though at times, what the researchers had to work with was only a tiny percent of the signal transduction of VEGF. Together with filt-1 (VEGF receptor 1) and flk-1 (VEGF receptor 2), neuropilin (NP) is frequently described as being involved in the neuroprotective effects of VEGF. The direct and indirect neuroprotective effects of VEGF, including various signaling pathways and the neurogenesis induced by this factor, are discussed in the context of the newly emerging insights into the biological mechanisms of VEGF and closely related, interacting molecules [7].

Leukocytes, particularly neutrophils and macrophages, persist in the surrounding tissue and secrete various proteases, including matrix metalloproteinases (MMPs) and serine proteases [8]. Excessive accumulation of these enzymes interferes with matrix remodeling [9]. Agents that inhibit proteases are thought to benefit wound healing [10]. Another feature of some chronic wounds is the reduction or absence of angiogenesis, which prevents nutrients from accessing the newly formed tissue [11]. Existing technologies to improve wound healing chronic wounds are initially managed by treatment comprising eschar debridement, an antibiotic treatment where appropriate, and regular dressing [2]. Other dressings may also be used, such as hydrogels, hydrocolloids, or alginates. Venous ulceration is treated by compression therapy, whereas arterial or diabetic ulcers require regular dressing changes. Pressure sores are encouraged to heal by relieving pressure at the injury site. Some other physical devices, such as laser treatment, hyperbaric oxygen, and electrical stimulation for arterial ulcers, are also used to promote wound healing [2, 12, 13].

The use of tissue-engineered skin, such as Dermagraft or Apligraf, is an option for wounds that are unresponsive to such interventions. This therapy prevents bacterial infection and allows the wound to heal by normal reparative processes [14, 15]. Using such skin replacements to accelerate wound healing depends on the availability of an existing vascular supply in the existing wound. Another approach to wound healing involves the administration of growth factors/cytokines, which have been shown to accelerate cell proliferation in vitro to promote wound healing in some animal models. These include IL-1, platelet-derived growth factor (PDGF), EGF, VEGF, TGF-β, and bFGF [2]. Procuren (Curative Technologies), an autologous platelet releasate, contains at least five growth factors that aid in forming granulation tissue and re-epithelialization. This autologous growth factor mix has achieved some success in human subjects with ulcerated limb lesions [16]. However, on the whole, results from most clinical trials using growth factors/cytokines have been disappointing. For example, EGF failed to heal venous stasis ulcers, and IL-1 failed to treat pressure sores effectively [2]. Similar results were reported using bFGF [17]. The reason for the lack of efficacy is not certain but may relate to the multifactorial effects, some undesirable for healing, of growth factors/cytokines.

The patent by Jackson utilizes activated protein C (APC) to assist with wound healing. According to the patent, it has been reported that APC additionally acts as an anti-inflammatory agent and directly activates the protease gelatinase A [18, 19].

Many different cell types, including smooth muscle cells, fibroblasts and endothelial cells secrete gelatinase A. By degrading the collagens in the basement membrane and allowing cells to invade the stroma, gelatinase A plays a vital role in physiological remodeling and angiogenesis [21]. Further, APC is also able to promote the regeneration of endothelial cells after wounding in vitro, stimulate re-epithelialization, fibroblast invasion, and angiogenesis in a chicken embryo and enhance wound healing in a rat wounding model. When taken together with the abovementioned anticoagulating, anti-inflammatory, and Gelatinase A-activating functions, these functions strongly indicate that APC, functional fragments thereof, and the precursor of APC (i.e. protein C) is/are useful for the treatment of wounds and, particularly, slow-healing wounds.

Another patent describes a topical composition for treating cutaneous wounds using whole colostrum. Growth factors, immune factors, enzymes, and micro- and macro-nutrients are bioactive in colostrum and associated with wound healing. Some growth factors mentioned include epidermal, fibroblast insulin-like I & II, transforming alpha & beta, and platelet-derived nerve. Some immune factors include immunoglobulins, lactoferrin, cytokines, proline-rich polypeptide, and lactalbumins. Some enzymes include lysozyme, peroxidase, proteases, and xanthine oxidase. Micro- and macro-nutrients include carbohydrates, amino acids, vitamins, and minerals.

Human plasma fibronectin is described in a patent [23]. In this patent, the formulations provided a slow release and increased contact time of fibronectin to the wound site leading to effective absorption. Fibronectin is involved in all stages of wound healing. Some of the biological activities of fibronectin related to wound healing include cell recruitment, opsonization of particulate debris, and promotion of wound contraction [24]. In addition, topically applied fibronectin has been useful in increasing the rate of wound healing, such as in corneal wounds [25, 26] and leg ulcers [27]. The patent focuses on a gel and cream formulation.

Patent [28] describes topical or transdermal delivery of a composition promoting wound healing that includes nitric oxide and/or peptides such as thyrotropin-releasing hormone (TRH) and/or GnRH (gonadotropin-releasing hormone).

The growth factor approved by the US Food and Drug Administration for treating diabetic foot ulcers is recombinant PDGF-BB (becaplermin), which comes in as a topical cream. PDGF-B is known to be a potent mitogen and chemotactic agent for stromal cells and may act to increase wound vascularization by stimulating angiogenesis.

US Patent [29] utilizes Angiopoietin-like protein 4 (ANGPTL4) to assist with wound repair. ANGPTL4 has been shown to play a context-dependent role in angiogenesis and vascular permeability [30-32]. The deficiency in ANGPTL4 in mice resulted in delayed wound re-epithelialization, reduced matrix proteins expression, increased inflammation, and impaired wound-related angiogenesis [33, 34].

A specific area of wound healing includes neuroprotection and neural regeneration of damaged axons and nervous tissue. When axons of the mammalian peripheral nervous system (PNS) are severely damaged (i.e., in compression or transection injuries), several phenomena may occur. First, if left unmodified, the distal stump will nearly always degenerate (Wallerian degeneration), This degeneration is associated with concomitant chromatolytic changes proximally in the perikaryon. If damage to the nerve is sufficiently severe, the axons in the proximal portion will degenerate, followed by the degeneration and (usually) death of the perikaryon. If the damage to the nerve is less severe and left unmodified, a number of biochemical changes begin to occur in the remaining proximal portion. These proximal changes involve a complex series of responses of the cell body to the injury, which seem to prepare the intact portion for regeneration. Such changes include alterations in axonal transport characteristics, protein processing, and nucleic acid synthesis. Morphologically regenerating growth cones from the damaged proximal stump often appear, and the axons will begin regenerating from the proximal stump towards the original target region.

Such damage is likely to produce collateral sprouting from neighboring axons that are not as severely injured and did not undergo Wallerian degeneration. The newly growing neurites probably use the degenerated distal segment as a guide to the denervated target area. Presumably, the reactive Schwann cells provide the communicative means for regeneration to the target by providing diffusible growth-promoting factors and by providing a suitable growth surface established by the plasma membrane and/or basal lamina or extracellular matrix. These morphological and biochemical changes are primarily dependent upon several factors, including the severity and location of the injury depending upon the proximity to the perikaryon, the size of the axons injured, and the species involved. For example, higher vertebrates have less capacity to regenerate the peripheral nervous system (PNS) axons effectively.

In the central nervous system (CNS), the unaided attempts at regeneration often are quickly aborted by the body, resulting in a completely degenerated proximal stump and perikaryon.

Trophic substances from nearby tissues may have a greater trophic potential than the intended target tissue. As a result, the gap length of an injury has been shown in the prior art to be a primary factor in determining the success of functional regeneration. This lack of target specificity has been suggested in the literature as the underlying cause of the frequent formation of neuromas and inappropriate contact with other tissues. Thus, significant improvements have been observed after neural anastomoses have been made. The current method of choice in the neurosurgical repair of damage to peripheral nerves is simple anastomosing of the cut end of the nerve.

Many studies have been performed to determine nerve and/or axon regeneration after neuronal tissue injury. Axons damaged by traumatic injuries often cannot regenerate spontaneously in the adult central nervous system. Injuries to the peripheral nervous system cause neuropathies that lead to weakness and paralysis, poor or absent sensation, unpleasant and painful neuropathies, and impaired autonomic function [35]. The peripheral nervous system has some regenerative capacity.

### Background of Existing Composition and Formulations

The gel formulation comprises a water-soluble, pharmaceutically acceptable polymer which is prepared from an effective amount of fibronectin. Examples of such compounds include: vinyl polymers, e.g., polyacrylic acid; polyoxyethylene-polyoxypropylene block copolymers, e.g., poloxamer; and cellulose derivatives, e.g., hydroxypropylcellulose (HPC). The polymer provides viscosity values between 50,000 and 1,000,000 cps at room temperature. The cream formulation is prepared from a commercially available cream base, i.e., Schering base (Schering Canada Inc., Point-Claire, Quebec), which has viscosity values between 60,000 to 80,000 cps at room temperature. The present invention's slow-release system of gel formulations provides extended fibronectin release to the wound site. This property of these formulations permits less frequent application to the wound resulting in less disturbance to the healing process. Such formulations maintain fibronectin delivery for up to 24 hours. The preferred embodiment, according to kinetic data, prefers a twice-a-day therapeutic schedule.

A patent describes a wound healing composition comprising of an aqueous mineral oil emulsion that is readily spread able and film-forming and, upon application to moist skin or mucosal surface, forms a stable, coherent layer.

The composition can take the form of a gel, a cream, a lotion, an ointment, a solution, a solid, an adhesive patch with a reservoir (enabling a slow release of the composition) etc., that can be rubbed or sprayed onto the affected area including skin, subcutaneous tissue, muscle, fascia, blood vessels, and nerve requiring regeneration or healing.

Goals for wound repair include accelerated wound closure with minimal scar formation.

Several studies measuring the efficacy of epidermal growth fact (EGF) in wound healing have been undertaken with mixed results. Thornton et al. (1982) applied EGF topically to scald burns in rats [37]. Only an insignificant healing advantage over control was seen. Niall et al. (1982) reported an enhancing effect on wound healing in mice upon topical administration of EGF [38]. Brown et al. (1986) studied the activity of EGF topically applied to wounds in miniature pigs [39]. They report that it increased the rate of epithelialization of split-thickness wounds in vivo. In a fourth paper, Buckley et al. (1985) studied the effects of EGF upon slow release from subcutaneously-implanted sponges in rats [40]. They conclude that the sustained local presence of EGF accelerates the process of wound healing.

Studies by Lawrence et al. (1986) and Sporn et al. (1983) suggest an acceleration of wound healing in rats upon administration of transforming growth factor (TGF) [41, 42], and in Lawrence et al. (1986), a further enhancement upon the combined use of TGF, EGF, and platelet-derived growth factor (PDGF) [41]. In addition, Shultz et al. (1987) reported that topically applied transforming growth factor-alpha (TGF-a) and vaccinia growth factor (VGF) in antibiotic cream accelerated epidermal regeneration in partial thickness dermal burns (second degree) on the backs of pigs [43].

Recently, interest has been generated in the IGFs as described in the patent [44], insulin-like growth factor-I (IGF-I), also termed Somatomedin C, and insulin-like growth factor-II (IGF-II). Froesch et al. (1985) report that the major effects of IGFs are on the growth of cells of mesodermal origin and differentiation [45]. They further report that the constant high levels of IGF in the bound form in serum may serve several functions: replacement of dying cells, repair mechanisms, matrix synthesis, and perhaps a constant stabilization of cells, keeping them from transforming and dedifferentiating [45].

### Background of Prior Art in Nerve Regeneration

Simple anastomosing will not be sufficient in those circumstances where damage and degeneration are so extensive that the distance between the remaining proximal and distal stumps is excessive. An alternative solution employs a structure or “bridge” across the gap length from the cut proximal stump to either the distal portion of the nerve or the target tissue itself. In animal studies, the various materials used to bridge the gap include peripheral nerve grafts, mesothelial chambers, and millipore and silicone tubes.

Of particular interest are the artificially produced and commercially available “nerve cuffs’ or “nerve guide tubes,” which are implanted and extend between the stumps. The nerve guide tubes are generally made of either silicon or bioresorbable substances. The nerve guide tubes can be filled or coated with a growth-supporting matrix such as laminin that promotes neural growth over greater distances than the unmodified nerve guide tube alone.

Want T et al. showed beneficial effects of branched-chain amino acids on the neuronal survival and axon regeneration of retinal ganglion cells after optic nerve transection through the mammalian target of rapamycin (mTOR) pathway [46]. Nucleic acid therapy can improve regeneration by enhancing the intrinsic growth ability of neurons and overcoming the inhibitory environment that prevents neurite outgrowth. These nucleic acids modulate gene expression by over-expressing the neuronal growth factor or silencing growth-inhibitory molecules [47]. Another study measured neuronal regeneration by studying the olfactory nerve. Dysfunction of this nerve causes losing the sense of smell. The study combined the delivery of two growth factors, vascular endothelial growth factor, and platelet-derived growth factor, to increase the number of mature olfactory neurons [48]. In addition, Curcumin's neuroprotective activity was studied. The effects of local and continuous treatment using low curcumin doses were investigated for nerve regeneration after rat sciatic nerve crush. The curcumin treatment increased the expression of compact myelin proteins, myelin sheath thickness, and increased motor and sensitive nerve conduction velocity. Furthermore, Curcumin treatment reduced the production of reactive oxygen species (ROS) (notably produced by macrophages), lipid peroxidation, and increased expression of transcription factor Nrf2. This antioxidant capacity is likely to contribute to the beneficial effects of curcumin after SNC injury [49].

Advanced conduit design and fabrication techniques have made it possible to fabricate autograft-like structures in the NGCs with incredible precision. To this end, strategies involving biopolymers, cells, growth factors, and physical stimuli have been developed over the past decades and have led to the development of varying NGCs, from simple hollow tubes to complex conduits that incorporate one or more guidance cues [35]. One study combined using a nerve conduit and stem cells to enhance the recovery of the recurrent laryngeal nerve after injury. The results revealed that the laminin-chitosan-PLGA nerve conduit combined with Schwann and neural stem cells was able to promote nerve regeneration (P<0.05), and its effect was superior to those of the autograft (P<0.05) [50].

Non-pharmacologic agents have been used for nerve regeneration. This includes electrical stimulation. One study evaluated the regenerative effects of implanted electrodes with different contacts in the resected sciatic nerve. This method has shown increased sciatic functional index, increased amplitude of compound muscle action potential, increased motor nerve conduction velocity, and reduced muscular atrophy [51]. Another study on peripheral nerve injury showed that electrical stimulation combined with neural crest stem cells significantly enhanced nerve regeneration after injury and repair. The findings were comparable to autograft treatment [52].

Following injury, an axon can use one of several different strategies to re-join with its target tissue. It first needs to initiate regrowth, which can originate from the tip of the severed axonal end still attached to the cell body, a branch extending out from this fragment, or a new axon derived from the soma. To reach its target, the axon can navigate along the entire length of its original pathway or use a different, ectopic route. Due to the extensive regrowth that may be required, regrowing axons can instead make functional connections with other neurons in the vicinity, taking a similar route or with newly born neurons after cellular migrations into the damaged zone, thereby shortening the growth requirements. Peripheral nerve injuries causing major gaps between segments are routinely repaired with autologous nerve grafts [53]. An alternative approach, especially for severe injuries, is end-to-side neurorrhaphy, in which a transected distal nerve stump is joined to the trunk of an adjacent intact donor nerve [54]. This technique has been used for various peripheral nerve injuries, but mixed results and a lack of randomized clinical trials have limited its use [54]. Combining this approach with the application of an endothelin-B receptor agonist such as sovateltide (IRL-1620) may aid functional recovery and expand the clinical usefulness of neurorrhaphy approaches.

Growth Factors (GF) (e.g., epidermal, fibroblast, insulin-like I&II, transforming O&B, platelet-derived, nerve), immune factors (e.g., immunoglobulins, lactoferrin, cytokines, proline-rich polypeptide, lactalbumins) enzymes (e.g., lysozyme, peroxidase, proteases, Xanthine oxidase) micro- and macro-nutrients (e.g., carbohydrates, amino acids, vitamins, minerals) have also been proposed for treating wounds and repairing damaged nerves.

Background of Endothelin Analogs Associated with Wound Healing

Many current compositions exist to enable the healing of tissue structures. Endothelin has been reported to enhance many cascades that would be effective in wound healing. A study by Qin D. et al. investigated the role of endothelin-1 (ET-1) in the proliferation, migration, and secretion of extracellular matrix (ECMs), such as type I collagen and fibronectin, in retinal pigment epithelium (RPE) cells in vitro. Results illustrated that ET-1 promoted the proliferation, migration, secretion, and secretion of ECMs through the protein kinase B (Akt) and extracellular signal-regulated kinase (Erk) signaling pathways. This suggests that ET-1 may play a vital role in the development of proliferative vitreoretinopathy (PVR). PVR in turn represents an excessive wound-healing response via ECM molecules [55].

Endothelin has also been studied in hepatic wound healing. For example, in a study by Khimji et al. (2011), it was found that ET-1 is produced in increased amounts, and the cellular source of ET-1 shifts from endothelial cells to stellate cells during liver injury. This sets a feedback loop that accentuates further activation, stellate cell proliferation, and production of ECM proteins [56].

Another study by Lagares et al. (2010) showed that endothelin 1 contributes to the effect of transforming growth factor B1 (TGFB1) on wound repair and skin fibrosis. TGFB1 induced ET-1 expression in human dermal fibroblasts through Smad- and activator protein 1/JNK-dependent signaling. The ability of TGFB1 to induce the expression of profibrotic genes was dependent on ET-1. Adenovirus-mediated overexpression of TGFB1 and ET-1 in mouse skin was associated with accelerated wound closure, increased fibrogenesis, and scarring [57]. According to this study, an ET-1 receptor antagonist, such as bosentan, may represent a useful therapeutic tool in treating excessive scarring and fibrosis-related diseases [57]. Another study evaluated the process ET-1 has in wound healing. It showed that skin wound healing was accelerated when ET-1 was not attenuated via bleomycin. ET-1 mice showed earlier granulation tissue and re-epithelialization [58].

Endothelin has been involved in neurogenesis and neuroprotection. According to a study, an endothelin B receptor agonist given in an ischemic stroke-induced rat was compared to the control, where no drug was given. The endothelin B receptor agonist rats showed an increased number of blood vessel formations labeled with vascular endothelial growth factor, better preserved the neuronal numbers, and increased the expression of nerve growth factor [59-62].

### BRIEF SUMMARY

The aspects of the disclosed embodiments comprise a novel composition comprising an Endothelin B agonist to enhance wound healing of tissue that includes superficial and deep tissue structures including, but not limited to, epithelium, subcutaneous tissue, muscular tissue, fascia, bone, and neurovascular tissue. Specifically, the invention aims to use this composition to treat patients with acute or chronic wounds, including diabetic foot ulcers. In addition, this invention is geared towards a composition that assists with neural tissue regeneration after an acute or chronic neural injury, such as a nerve transection that occurs during surgery. In preferred embodiments, endothelin B agonists may have various compositions, including, but not limited to, bioactive factors, stem cells, proteins, and elements to enhance tissue recovery. Local drug delivery of the preferred embodiments may be topical or placed in deep tissue intraoperatively or injected. Systemic delivery may be an option for diffuse cases of tissue injury. These delivery systems include intravenous, intramuscular, and oral forms.

### BRIEF DESCRIPTION OF DRAWINGS

The patent or application file contains at least one drawing executed in color. Copies of this patent or patent application publication with color drawing(s) will be provided by the Office upon request and payment of the necessary fee.

These and other features, aspects, and advantages of the present invention will become better understood with regard to the following description, appended claims, and accompanying drawings where:

FIG. 1. Illustrates a Chromatogram of IRL-1620 (Retention Time 5.093)

FIG. 2. Illustrates Images of Cross-linking stage in Alginate Hydrogel films before Lyophilization.

FIG. 3. Illustrates Images of Completely Cross-linked Alginate Hydrogel films before Lyophilization.

FIG. 4. Illustrates Sterilization with UV radiations in Laminar flow.

FIG. 5. Illustrates Images of Lyophilized Hydrogel Alginate films.

FIG. 6. Illustrates FTIR of Hydrogel film

FIG. 7. Illustrates FTIR of Sodium Alginate

FIG. 8. Illustrates FTIR of IRL-1620

FIG. 9. Illustrates SEM images of Surface view of Alginate Hydrogel Films containing (a) Sodium Alginate—1% (b) Sodium Alginate—1.25% (c) Sodium Alginate—1.5% (d) Sodium Alginate—2%

FIG. 10. Illustrates Light Transmission property of Hydrogel Film

FIG. 11. Illustrates In vitro release pattern of IRL-1620 through Goat Skin from the formulation up to 26 hrs

FIG. 12. Illustrates In vitro release pattern of IRL-1620 through Goat Skin from the formulation up to 5 hrs

FIG. 13. Illustrates % Cumulative release pattern of IRL-1620 through Goat Skin from the formulation.

FIG. 14. Illustrates % Assay of Stability Samples of developed formulation at refrigerated (2-8° C.) and deep freeze (−20° C.) conditions.

FIG. 15. Illustrates Chromatogram of IRL-1620 (Retention Time 5.093)

FIG. 16. Illustrates Microscopic view of Niosomes F2 and F3

FIG. 17. Illustrates Entrapment Efficiency of different formulations

FIG. 18. Illustrates FTIR Spectra of IRL-1620

FIG. 19. Illustrates FTIR Spectra of Excipient (Soya-Lecithin)

FIG. 20. Illustrates FTIR Spectra of Niosome (F3)

FIG. 21. Illustrates Particle size distribution graph

FIG. 22. Illustrates Zeta Potential Distribution Peak

FIG. 23. Illustrates Niosome Gel G-3 (Used for In vitro release and Wound Healing studies in rats)

FIG. 24. Illustrates In vitro release pattern of IRL-1620 through Goat Skin from the formulation G-3.

FIG. 25. Illustrates % Cumulative release pattern of IRL-1620 through Goat Skin from formulation G-3.

FIG. 26. Illustrates % Assay of Stability Samples of developed formulation at room temperature and refrigerated condition.

FIGS. 27A, 27B, 27C, 27D, 27E, 27F, 27G, 27H: figures demonstrating effect of different treatment on inflammation, angiogenesis and fibroblastic proliferation in respective groups of excision model

FIG. 28A, 28B, 28C, 28D, 28E, 28F, 28G, 28H: figures demonstrating effect of different treatment on collagen deposition in respective groups of excision model

FIG. 29A, 29B, 29C, 29D, 29E, 29F, 29G, 29H: figures demonstrating effect of different treatments on VEGF expression in respective groups of excision model

FIG. 30A, 30B, 30C, 30D, 30E, 30F, 30G, 30H: figures demonstrating effect of different treatments on vWF expression in respective groups of excision model

FIG. 31: Figure demonstrating effect of different treatment on wound healing in different groups

FIG. 32A, 32B, 32C, 32D, 32E, 32F, 32G, 32H: figures demonstrating effect of different treatment on inflammation, angiogenesis and fibroblastic proliferation in respective groups of excision model

FIG. 33A, 33B, 33C, 33D, 33E, 33F, 33G, 33H: figures demonstrating collagen deposition in different groups of incision model

FIG. 34A, 34B, 34C, 34D, 34E, 34F, 34G, 34H: figures demonstrating VEGF expression in different groups of incision model

FIG. 35A, 35B, 35C, 35D, 35E, 35F, 35G, 35H: figures demonstrating vWF in different groups of incision model

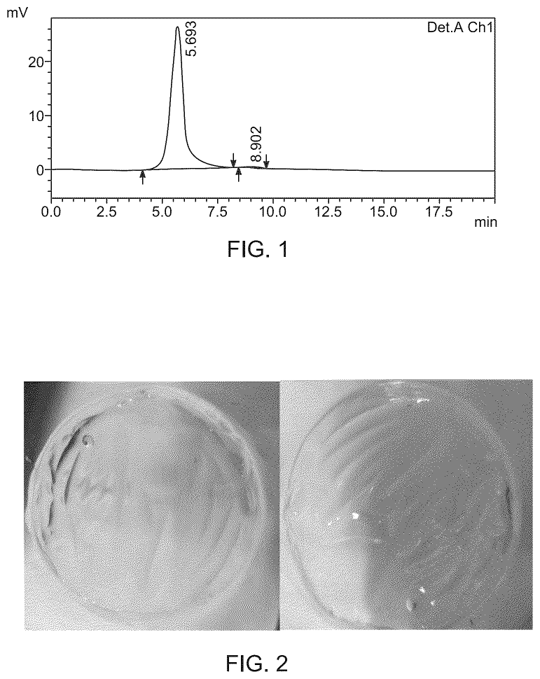

### DETAILED DESCRIPTION

A method to enhance wound healing of tissue that includes superficial and deep tissue structures including, but not limited to, epithelium, subcutaneous tissue, muscular tissue, fascia, bone, and neurovascular tissue is disclosed. Specifically, the invention aims to use this composition to treat patients with acute or chronic wounds, including diabetic foot ulcers. In addition, this invention is geared towards a composition that assists with neural tissue regeneration after an acute or chronic neural injury, such as a nerve transection that occurs during surgery. Endothelin B receptors are important in neuroprotection and neural regeneration, along with enhancing the physiologic wound healing cascade. The present invention is directed toward tissue healing using endothelin B receptor analogs such as N-Succinyl-[Glu9, Ala11,15] endothelin 1 (sovateltide, IRL-1620), BQ-3020, [Ala1,3,11,15]-endothelin, sarafotoxin S6c, and endothelin 3. Sovateltide (IRL-1620) enhanced cerebral blood flow, increased angiogenesis, neurogenesis, reduced apoptosis, and mitochondrial fission. IRL-1620 induced neural progenitor cell differentiation into mature neurons. Anti-apoptotic, anti-inflammatory, and anti-oxidant activity of IRL-1620 limits the death of cells, and it also promotes the formation of new neurons (neurogenesis) and new blood vessels (angiogenesis), enhancing the healing of wounds and regenerating nerves.

### Definitions

As used herein, the term “an amount sufficient to” refers to the amount that enables the achievement of the intended effect, for example, to increase the quantity of fibroblast in a wound bed or improve conduction through a neural pathway. Such an amount may be determined through various assays known in the art based on the intended effect. As used herein, the terms “applying” or “administering” refer to all means of introducing the specified agent, composition, or force to the specified region or subject. “Administration” or “application” can be effected in one dose, continuously or intermittently throughout treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern selected by the treating physician. Suitable dosage formulations and methods of administering the agents are known in the art. Route of administration can also be determined, and the method of determining the most effective route of administration is known to those of skill in the art and will vary with the composition used for treatment, the purpose of the treatment, the health condition or disease stage of the subject being treated, and target cell or tissue. Non-limiting examples of the route of administration include oral administration, nasal administration, inhalation, injection, and topical application. Administration can be for use in industrial as well as therapeutic applications. As used herein the term “biodegradable” is used herein to describe substances, such as polymers, compositions, and formulations, intended to degrade during use. Biodegradable substances may also be “biocompatible,” i.e., not harmful to living tissue. Non-limiting exemplary biodegradable substances include poly(lactic acid) (PLA) and poly(lactic-coglycolic) acid (PLGA), optionally pegylated.

The term “neural tissue” refers to tissue that improves the conduction of signals of motor and sensory nerve pathways. This may include neurons, nerve cells, glial cells, axons, astrocytes, microglial cells, ependymal cells, oligodendrocytes, enteric glia, satellite cells, and Schwann cells. Neural tissue also includes parts of the central nervous system, including the brain and spinal cord, along with the peripheral nervous system that regulates and controls bodily functions and activity.

As used herein, the term “tissue” refers to the tissue of a living or deceased organism or any tissue derived from or designed to mimic a living or deceased organism.

As used herein, the term “therapeutically effective amount” refers to a quantity sufficient to achieve the desired effect. In the context of therapeutic applications, the effective amount will depend on the type and severity of the condition at issue and the characteristics of the individual subject, such as general health, age, sex, body weight, and tolerance to pharmaceutical compositions. The skilled artisan will be able to determine appropriate amounts depending on these and other factors. In the case of an in vitro application, in some embodiments, the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of composition depending on the embodiment. The dose range of N-Succinyl-[Glu9, Ala11,15] endothelin 1 (IRL-1620, sovateltide) could be from 0.00001 to about 1 mg and may be administered once or multiple times in a day or in weeks or in months.

As used herein, the term “treating” or “treatment” includes preventing a disease, disorder, or condition from occurring in a subject predisposed to or having a disease, disorder and/or condition; inhibiting the disease, disorder or condition, e.g., impeding its progress; and relieving or reversing the disease, disorder, or condition, e.g., causing regression of the disease, disorder and/or condition. Treating a disease or condition may also include ameliorating at least one symptom of the particular disease or condition.

### Compositions

Aspects of the disclosure relate to a composition or an agent that regenerates tissue lost or damaged tissue. The basic composition includes Endothelin B receptor agonists with or without stem cells. In some embodiments, the basic composition may not include stem cells.

In some embodiments, the basic composition may be combined with growth factors and/or cytokines known to promote tissue regeneration. Neuroregenerative compounds include, but are not limited to, growth factors, an osmotic agent such as glycerol, a diuretic such as furosemide or ethacrynic acid, a steroid, transplanted stem cells, ferulic acid, arctigenin, espin isoform 1 polypeptide, neurotrophin selected from the group consisting of glial cell line-derived neurotrophic factor, brain-derived neurotrophic factor, neurotrophin-3, neurotrophin-4/5, nerve growth factor, vascular endothelial growth factor, and ciliary neurotrophic factor, insulin-like growth factor, Netrin1, thyroid hormone, hepatocyte growth factor, luteolin, neuropilin, epidermal growth factor, transforming growth factor-β, human plasma fibronectin, platelet-derived growth factor, and basic fibroblast growth factor. In addition, the embodiment may include Procuren (Curative Technologies), an autologous platelet releasate with at least five growth factors that aid in forming granulation tissue and re-epithelialization. This autologous growth factor mix has achieved some success in human subjects with ulcerated limb lesions.

In some embodiments, the basic composition may be combined with chronic wound healing strategies, including but not limited to eschar debridement techniques, antibiotic treatment, compression therapy, laser treatment, hyperbaric oxygen, and electrical stimulation. Dressings for such wounds may include hydrogels, hydrocolloids, or alginates and may be part of the embodiment.

In some embodiments, the basic composition may be combined with compounds that have been known to assist in wound healing, including activated protein C, which promotes the regeneration of endothelial cells. Another compound includes colostrum which also contains growth factors, immune factors, enzymes, and micro- and macro-nutrients that are present in colostrum that is bioactive and associated with wound healing. Angiopoietin-like protein 4 (ANGPTL4) is another compound to assist with wound repair. ANGPTL4 has been shown to play a context-dependent role in angiogenesis and vascular permeability.

In some embodiments, the basic composition may be combined with compounds that have shown neuronal survival, recovery, and regeneration, including branched-chain amino acids, nucleic acids that overexpress neuronal growth factors, and curcumin.

In some embodiments, the basic composition may be combined with immune factors, including immunoglobulins, lactoferrin, cytokines, proline-rich polypeptide, and lactalbumins.

In some embodiments, the basic composition may be combined with hormones, including thyrotropin-releasing hormone and gonadotropin-releasing hormone.

In some embodiments, the basic composition may be combined with enzymes and/or micro- and macro-nutrients, including lysozyme, peroxidase, proteases, xanthine oxidase, carbohydrates, amino acids, vitamins, and minerals.

In some embodiments, the basic composition may be combined with compounds that contain a degrative process that promotes wound healing, such as gelatinase A.

In some embodiments, stem cells used in the basic composition may include but are not limited to haematopoetic stem cells, embryonic stem cells, and stem cells from platelet-rich plasma. In some embodiments, stem cells may also be genetically modified as vectors to produce factors that promote the regeneration of damaged tissue, including neural tissue. Finally, in some embodiments, stem cells may be transplanted into the damaged tissue prior to administration of the Endothelin B receptor agonist.

In some embodiments, the basic composition may be combined with tissue-engineered skin such as Dermagraft or Apligraf. This therapy acts to prevent bacterial infection and allows the wound the chance to heal by normal reparative processes.

### Preferred Embodiment Preparation of Sovateltide (IRL-1620) Stock Solution

### Drug Substance

IRL-1620 (98.5% purity) manufactured and marketed by American Peptide Company, Inc. 1271 Avenida Chelsea Vista, CA 92081 USA. Sodium alginate, Glycerol, Calcium Chloride, and all the other excipients and solvents were purchased from Merck.

### Drug Analysis

### Analytical Method for IRL-1620 (Sovateltide)

HPLC method was used for the analysis of IRL-1620. Analysis was performed on gradient HPLC with 1% TFA in Water:and 0.1% TFA in Acetonitrile (55:45) as mobile phase. The mobile phase was prepared freshly every day. The mobile phase was filtered through a 0.2 mm membrane filter to remove any particulate matter, mixed and degassed by sonication before use, and found to be stable with no precipitation with time or decrease in temperature. The absorbance of IRL-1620 was fixed at 215 nm without any interference. The sensitivity of the detector was set at 0.01AUFS. Before injecting solutions, the column was equilibrated for at least 60 minutes with the mobile phase flowing through the system. Each solution was injected in triplicate, and the relative standard deviation (RSD) was required to remain below 1.0% on a peak area basis. The details of chromatographic conditions are as follows:

TABLE NO. 1
Chromatographic Conditions for Analysis of IRL-1620

| Parameters | Condition | |
|---|---|---|
| Mobile Phase | 0.1% TFA in Water:0.1% TFA in Acetonitrile | |
| | (55:45) | |
| Analytical column | XTerra (Waters) RP18 (150 mm × 4.6 mm) | |
| | chemically bonded to porous | |
| | 5 μm silica particles | |
| Diluent | Acetonitrile:Water 40:60, v/v | |
| Flow rate | 0.3 | mL/min |
| Injection volume | 20 | μl |
| Detection wavelength | 215 | nm |
| Run time | 20 | min |
| Retention time | 5.0 ± 0.5 | min |

### Preparation of IRL-1620 Stock Solution

Accurately 1.0 mg of IRL-1620 lyophilized powder was weighed and transferred into a 10 mL volumetric flask. The volume was made up with HPLC water to get the concentration of 100 μg/mL of IRL-1620 (IRL-1620 Stock-I). Next, an aliquot of 100 μl of the above solution (Stock-I) was transferred into a 10 mL volumetric flask, and the volume was made up with diluent to get the concentration of 1.0 μg/ml. The solution was labeled as IRL-1620 Stock-II and stored at −20° C. Stock solutions of IRL-1620 were used over the period of 30 days and stored properly at −20° C.

### Formulations and Modes of Administration

The formulations that include the basic composition that promotes wound healing and nerve regeneration is referred to as Endothelin B receptor agonist with or without stem cells.

In some embodiments, the basic composition that promotes wound healing and nerve regeneration may be synthesized in varying formulations and/or particles, as described below.

The composition may be formulated to take the form of a gel, a cream, a lotion, an ointment, a solution, a solid, an adhesive patch with a reservoir (enabling the slow release of the composition) etc., that can be rubbed or sprayed onto the affected area including skin, subcutaneous tissue, muscle, fascia, blood vessels, and nerve requiring regeneration or healing.

In some embodiments, the formulation may comprise a water-soluble gel polymer that is prepared from an effective amount of fibronectin. Examples of such compounds include vinyl polymers, e.g., polyacrylic acid; polyoxyethylene-polyoxypropylene block copolymers, e.g., poloxamer; and cellulose derivatives, e.g., hydroxypropylcellulose (HPC). The polymer provides viscosity values between 50,000 and 1,000,000 cps at room temperature. The cream formulation is prepared from a commercially available cream base, i.e., Schering base (Schering Canada Inc., Point-Claire, Quebec), which has viscosity values between 60,000 to 80,000 cps at room temperature. The slow-release system of gel formulations provides extended release of fibronectin to the wound site. This property of these formulations permits less frequent application to the wound resulting in less disturbance to the healing process. Such formulations maintain fibronectin delivery for up to 24 hours. The preferred embodiment in such a case, according to kinetic data, prefers a twice-a-day therapeutic schedule.

In some embodiments, the mode of delivery may comprise an aqueous mineral oil emulsion that is readily spread able and film-forming and, upon application to moist skin or mucosal surface, forms a stable, coherent layer.

In some embodiments, the mode of delivery may comprise medicated dissolvable sponges such as gel-foam. This may be implanted subcutaneously, at the wound bed, or during surgery prior to closure of the incisional defect.

For specific neural tissue regeneration, the mode of delivery of the preferred embodiment may employ the use of a structure or “bridge” across the gap length from the cut proximal nerve stump to either the distal portion of the nerve or to the target tissue itself. In animal studies, the various materials used to bridge the gap include peripheral nerve grafts, mesothelial chambers, millipore and silicone tubes. The nerve guide tubes may be made of either silicon or bioresorbable substances. The nerve guide tubes can be filled or coated with the compositions discussed for the preferred embodiment. A conduit design may be fabricated into autograft-like structures combining the composition of the preferred embodiment. The use of biopolymers, cells, growth factors, and physical stimuli may be incorporated into the conduits that enable one or more guidance cues. For example, a nerve conduit may be made of Laminin-chitosan-PLGA and supplanted with the composition of our preferred embodiment and stem cells to enhance the recovery of the injured nerve.

In some embodiments, the formulation comprises a biodegradable polymer. In further embodiments, the biodegradable polymer is poly(lactic-co-glycolic acid) (PLGA) or pegylated PLGA (PEG-PLGA). In some embodiments, the composition may include further additives, including but not limited to polyvinyl alcohol (PVA) or other known nanoparticle stabilizers. In some embodiments, the nanoparticle is magnetically responsive or includes a magnetically responsive particle. In some embodiments, the magnetically responsive particle is an optionally superparamagnetic iron oxide (SPION). Finally, in some embodiments, the nanoparticle may be further comprised in a solution, suspension, gel, or other formulation suitable for its delivery.

The embodiment may be formulated to facilitate the timing of the release; for example, a poorly water-soluble second agent (e.g., TIDE) could be encapsulated in the organic shell of ETBRA with or without stem cells loaded with the hydrophilic agent that regenerates damaged tissue.

Our preferred embodiment for topical drug delivery is a niosomal gel and a thin film hydrogel.

### Thin Film Hydrogel Embodiment

The hydrogel developed for experimental purposes was a thin film of calcium alginate hydrogel. The steps toward development were as follows:

### Materials and Methods

### Estimation of IRL-1620 in Hydrogel Samples by HPLC Analysis

About 10 mg of Hydrogel samples were weighed in a centrifuge tube. 10 ml of Phosphate Buffer (pH 7.4) was added to this tube and sonicated at 2-8° C. for 30 mins. It was then centrifuged at 5000 rpm at 5° ° C., and the supernatant was collected. Next, 0.5 ml of supernatant was withdrawn in a 10 ml volumetric flask, and the volume was made up with diluent (Acetonitrile:Water 40:60), v/v solvent. The samples were analyzed using the HPLC method.

### Preparation of Alginate-Based Films

Alginate films were prepared using the solvent-casting process (Pereira et al., 2013). Solution of sodium alginate (1.5%, w/v) was prepared by powder dissolution in distilled water under mechanical agitation at 600 rpm until a homogeneous solution was formed (Approximately 2 hours). During the preparation of the alginate solution, the plasticizer agent, glycerol, was added at a percentage of 15% (w/w) based on the weight of the alginate powder. IRL-1620 in 1 ml water was dispersed and agitated in an ice bath at 600 rpm. Then, the solutions were transferred to an ultrasound system (30 min) under a controlled temperature (2-8° C.). It was left to degasify for 12 h in a refrigerator at 2-8° C. Alginate neat films with glycerol were prepared by casting 5 mL of solution into petridishe (ø=5 cm). The films were left to set for 5 mins. Next, 2 mL of 5.0% (w/v) CaCl₂) aqueous solution was poured in the petridish and agitated slightly to disperse CaCl₂) evenly and to allow the crosslinking for 15 mins. Then, the hydrogel was washed with distilled water, sterilized in a laminar flow unit in UV radiations, and kept in deep freeze for 48 hours. The films were lyophilized using a Freeze dryer (Allied Frost) until a constant weight was achieved (DiMicco et al., 2007).

### Compatibility Studies

The compatibility between IRL-1620 and the excipients Sodium Alginate was evaluated using the FTIR peak matching method (Riaz & Ashraf, 2015). The instrument used was Bruker FTIR (Alpha-1) over the range of 4,000 cm⁻¹ to 650 cm⁻¹.

### SEM Studies

The surface morphology of the samples containing different concentrations of sodium alginate was investigated by scanning electron microscopy (SEM LEO43 SVP, Cambridge) (Hashem et al., 2013).

### Film Thickness

The film thickness was measured using a Vernier Calliper micrometer at five different positions of the film with 0.001 mm of accuracy. The result was expressed as a mean of the measurements±standard deviation (SD).

### Film Light Transmission and Transparency

Film light transmission and transparency were determined by spectrophotometry according to the method used by Norajit et al. Rectangular samples of the films (10 mm×35 mm) were cut, placed into a spectrophotometer cell, and their light barrier properties measured at wavelengths comprised between 200 and 800 nm, using air as a control. The transparency of the films was determined by Eq.

$$Transparency = {Abs}_{600} / x$$

where Abs₆₀₀ represents the value of absorbance at 600 nm, and x corresponds to the film thickness in millimeters (Norajit & Ryu, 2012).

### Water Solubility

It is widely recognized that water solubility and swelling are important properties for the characterization of biodegradable materials to be used in tissue regeneration and drug delivery applications.

To determine the water solubility, crosslinked samples of hydrogel alginate films were lyophilized and then immersed in 40 mL of distilled water and stirred at 120 rpm for 24 h. Afterward, the samples were withdrawn from the medium, the excess water was removed with filter paper, and their wet weight was immediately determined to calculate the water absorption. The samples were then dried in an oven at 37° C. until constant weight to determine the solubilized mass.

Water absorption and water solubility were determined by the following equations:

$$Water absorption ( \% ) = [ ( Ww - Wd ) / Wd ] \times 100$$

$$Water solubility ( \% ) = [ ( Wi - Wf ) / Wi ] \times 100$$

Where, Ww represents the wet weight of the films, Wd corresponds to the dry weight of the films, Wi represents the initial dry weight of the film before immersion in distilled water, Wf corresponds to the dry weight of the film after immersion (Brown et al., 2011).

### Swelling Behavior

To determine the swelling behavior of the hydrogel film, a small rectangular film was cut in the dimension of 1×1 cm and weighed. The film was then placed in 20 ml phosphate buffer saline (pH 7.4) in a Petri plate. The hydrogel was taken out of the buffer after 30 min and reweighed. Thus, the swelling behavior was measured as a ‘% swelling index

$$\% swelling index = [ ( Wh - Wd ) \times 100 ] / Wd$$

Wh is the weight of the product after 30 min of hydration, and Wd is the weight of the dried product (S. W. Kim et al., 1992).

### Biodegradation Studies

A small section (2×2 cm) of the Film was placed in 25 ml of phosphate buffer at room temperature for 7 days. The dissolution of the film was observed visually.

### In-Vitro Drug Release Studies

In-vitro dissolution studies for hydrogel film were carried out through goat skin. Fresh goat skin was collected from the local slaughterhouse. It was cleaned, and hairs were shaved off. The permeation of IRL-1620 was assessed using Franz diffusion Cell at 100 rpm in 50 ml of phosphate-buffered saline 7.4 pH as a dissolution media, maintained at 37±5° C. 0.5 ml aliquots were withdrawn at the specified time intervals of up to 48 hours, and the concentration of IRL-1620 was analyzed using HPLC. An equal volume of fresh media was replaced after each sampling to maintain the constant volume (Thu et al., 2012). The samples were diluted with 0.5 ml of Acetonitrile:Water (40:60, v/v), and HPLC analysis was performed.

### Stability Studies

The stability of Hydrogel film formulation was evaluated by keeping in sealed glass vials and storing them in three different storage conditions, that is, deep freeze (−20° C.), refrigeration temperature (2-8° C.), and room temperature for a period of 45 days (Sabale & Vora, 2012). The samples were withdrawn at different time intervals over a period of study, and a % assay of the film was performed using HPLC.

### Results

### Drug Analysis

The developed high-performance liquid chromatographic (HPLC) method for the quantification of IRL-1620 was simple and sensitive. Separation was carried out on the reversed-phase C18 column (250 mm×4.6 mm), and the column effluent was monitored by a UV detector at 215 nm. The study found the method to be precise, accurate, and specific. This method was successfully applied for entrapment efficiency, drug release, and stability studies (FIG. 2).

### Preparation of Hydrogel Film

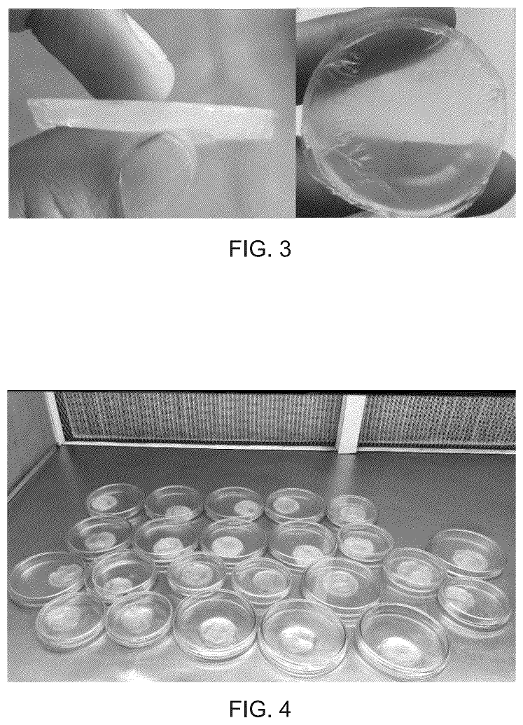

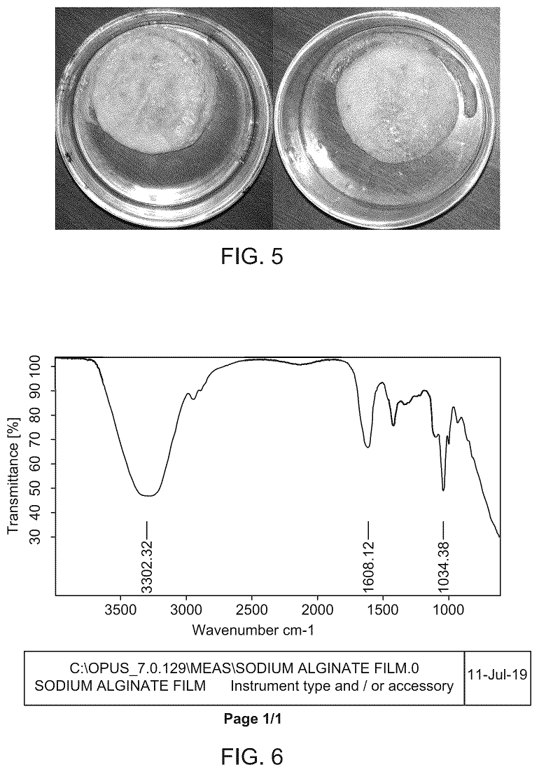

Transparent films were obtained with the addition of Calcium Chloride solution due to the formation of calcium alginate (FIG. 2). The films were allowed to harden for 15 minutes, and hardened homogeneous continuous, flexible films of hydrogel were obtained (FIG. 3). These films were deep-frozen and lyophilized. After lyophilization, thin white films of hydrogel were obtained (FIG. 5). It had a smooth surface, was flexible, and was not brittle in nature.

### Compatibility Studies

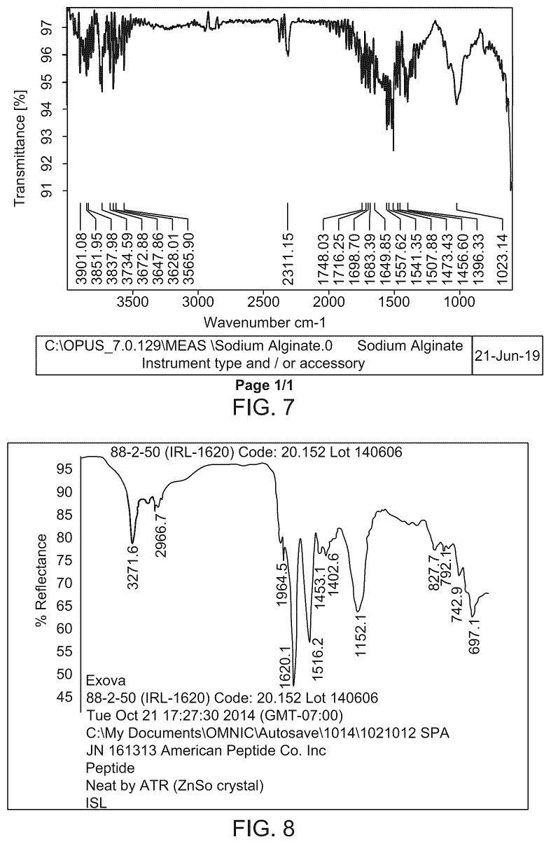

There was no appearance or disappearance of peaks of the IR spectra in the drug and polymer mixture, which confirmed the absence of any chemical interaction between the drug and sodium alginate, as shown in FIG. 6-8.

### Surface Morphology Studies by SEM

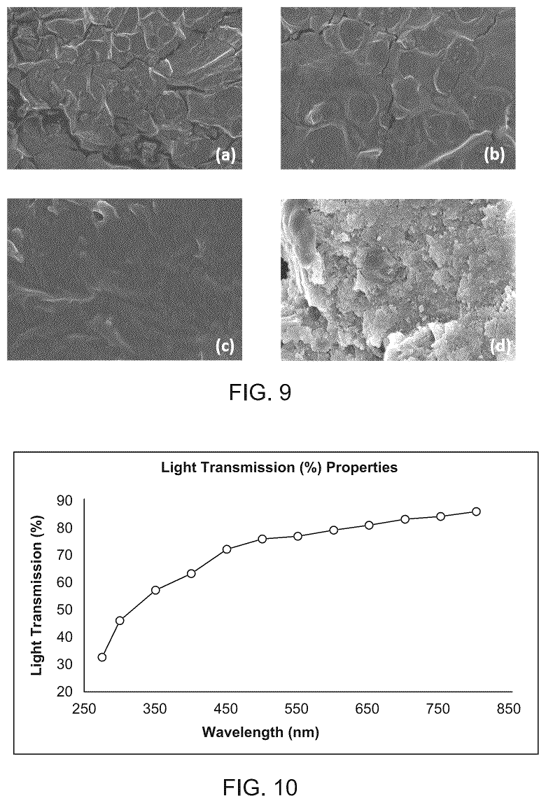

The Surface morphology of the Lyophilized Hydrogel films was performed using SEM studies. It was also performed for different concentrations of Sodium Alginate films (FIG. 9). The best morphological view was (FIG. 9 (d)), and thus, it was used for further studies. The 1.5% sodium alginate films had smooth and even textures depicting the fine crosslinking of the alginate moieties. The 1% and 1.25% concentrations gave brittle films with many cracks. A higher concentration (2%) of sodium alginate showed the formation of a rough film.

### Film Thickness

The developed Hydrogel film presented a smooth surface, high malleability, and thickness comprised in a range of 110 to 120 μm (Table. 2). The thickness of the films affects the in vivo dissolution in the wound area; thus, a low concentration of sodium alginate was selected (Tokarev & Minko, 2010). It was also enough to maintain the mechanical strength and reduce fragility of the films.

TABLE NO. 2
Results of Film Characterization studies

| Parameters | Results |
|---|---|
| Film Thickness | 110-120 μm |
| Film Transparency | 1.80 |
| Water Solubility at 37° C. (Solubilized Mass %) | 8.32 |
| Water absorption after 24 hrs (%) | 146.81 |

### Film Light Transmission and Transparency

The property of light transmission and transparency are important parameters to be assessed to determine the quality and effectiveness of the film in topical wound healing (Norajit & Ryu, 2012). The low concentration of IRL-1620 did not affect the transparency of the films.

### Water Solubility

The water solubility and swelling behavior of hydrogels were investigated to assess the behavior of the films when they are in permanent contact with biological fluids or tissues. The water solubility in 24 hrs was around 8.32% (Table 2). Thus, the film could be placed in open wounds for a longer period of time.

### Swelling Behavior

The swelling behavior of the hydrogel film was found to be 146.8%; this high swelling ability of the hydrogels can be credited to the hydrophilic functional groups such as NH₂— and COO— from Alginate polymer [63]. Thus, the hydrogel film developed can be used as a wound-healing agent with a high capacity to absorb the aqueous solution. In addition, it further helps in the absorption of wound exudates.

### Biodegradation Studies

The samples were observed daily for dissolution. The film swelled immediately, and the size of the film reduced gradually every day. Finally, a homogeneous solution was formed on day 7 of the study. Thus, it took 7 days for the film to degrade and release alginate in phosphate buffer.

### In-Vitro Drug Release Studies

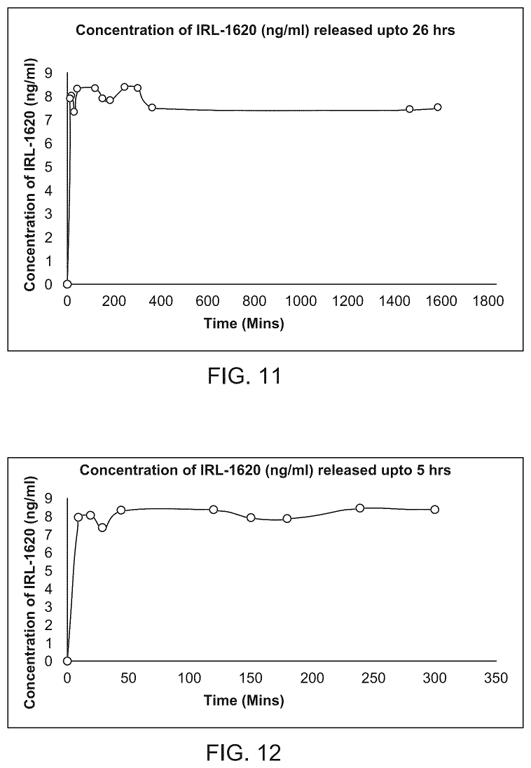

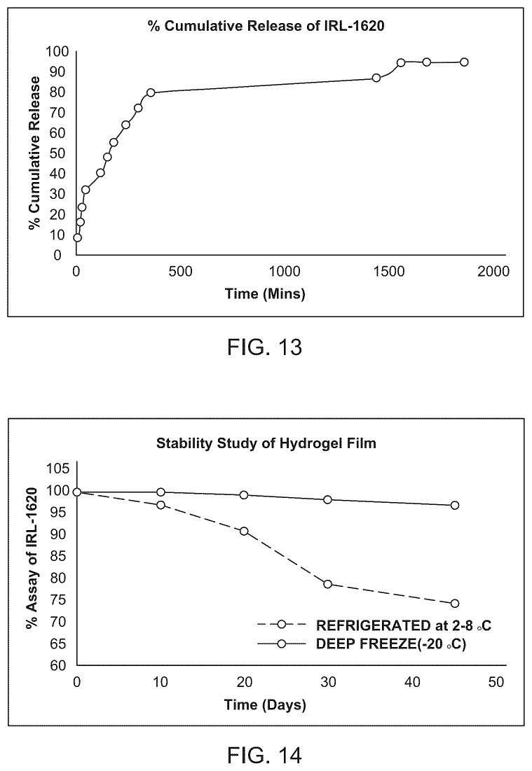

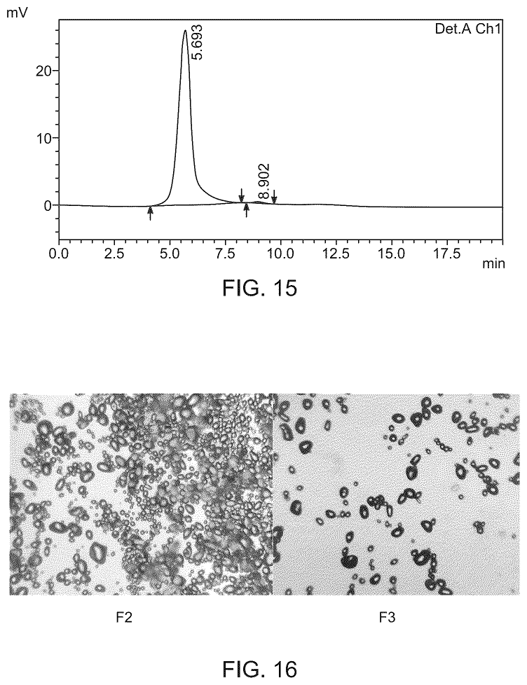

In vitro studies of Alginate Hydrogel film, formulations were carried out in PBS pH 7.4 using Franz diffusion Cell through goat skin, and results were shown in FIGS. 11 & 12. It was observed from the data that in vitro drug release of IRL-1620 from hydrogel film formulation was sharply increased till 5 hours, followed by a maintained concentration for around 24 hours with a sharp decrease in the release after 26 hrs. The cumulative release of IRL-1620 from the formulation was 86.54% after 24 hours (FIG. 13).

### Stability Studies

Stability studies were carried out by keeping the alginate film formulation for 45 days at different storage conditions. The samples kept at room temperature showed a change in their physical appearance. The films' color changed after 15 days of storage; thus, the films can be considered unstable at room temperature. The sample in the refrigerator and deep freeze showed physical stability, and thus assay of IRL-1620 was done using the HPLC method, and the results are shown in FIG. 14.

It was observed from the results that there is a decrease in drug content at 2-8° C., whereas, in the case of deep freeze (−20° C.), no significant change was observed. Thus, the developed hydrogel formulation was found to be stable at the end of the study in deep freeze (−20° C.).

### Conclusion

Alginate-based hydrogel dressings were successfully developed using glycerol as a plasticizer and calcium chloride as a cross-linker by solvent casting method. The 1.5% sodium alginate concentration provided hydrogel films of smooth surface and good mechanical strength. The swelling factor and water solubility of the hydrogel prove it suitable for wound healing for a longer period. The release of IRL-1620 was found to be up to 24 hours; however, alginate film stays in contact with the tissues and can continue wound healing due to water retention in the affected area. The formulation was found to be stable at −20° C.

### Niosome Gel Embodiment

Another alternative to our preferred embodiment is a niosomal gel which have both hydrophilic and hydrophobic properties. They allow for prolongation of the release of entrapped drugs and penetrate the layers of the epithelium due to the presence of cholesterol [64].

### Estimation of IRL-1620 in Niosomes Samples by HPLC Analysis

Accurately weigh about 1.0 mg of niosomes in a 10 ml volumetric flask. Add 0.5 ml of butanol and shake to completely moisten the niosomes. It will rupture the cholesterol and soyalecithin membrane. Diluent solvent and make up the volume to 10 ml. The samples were analyzed using the HPLC method.

### Estimation of IRL-1620 in Niosome Gel Samples by HPLC

Accurately weighed 100 mg of Niosome Gel, dispersed in 10 ml of phosphate buffer (pH 6.8), and centrifuged at around 3000 rpm. The supernatant was removed, the sedimented residue was treated with 0.5 ml butanol, and the volume was made up to 10 ml using Acetonitrile:Water 40:60, v/v. The samples were analyzed using the HPLC method.

### Preparation of Niosomes

IRL-1620 was dissolved in ethanol and used for the preparation of Niosomes. Niosomes complex in the ratios of (1:1, 1:2, 1:3, 1:4, and 1:5 Drug and soyalecithin) were prepared using ethanol as a reaction medium; cholesterol concentration was constant throughout. Soya lecithin and cholesterol were dissolved in 10 ml (Pando et al., 2013) of dichloromethane and added drop by drop to 5 ml of ethanol solution containing IRL-1620 (20 μg/ml) with continuous stirring and sonicated for 15 min. The resultant mixture was evaporated under vacuum at 2-8° C. The residue (niosomes) was placed in a desiccator. The niosomes were stored at 2-8° C.

TABLE NO. 3
Niosome Combinations for IRL-1620

| | Formu- | Ingredients | | | | |
|---|---|---|---|---|---|---|
| | lation | Drug | Soylecithin | Cholestrol | Dichloro- | |
| | Code | (μg) | (mg) | In (mg) | methane | Ethanol |
| Ethanol | F1 | 100 | 10 | 10 | 2 ml | 10 ml |
| method | F2 | 100 | 20 | 10 | 2 ml | 10 ml |
| | F3 | 100 | 30 | 10 | 4 ml | 10 ml |
| | F4 | 100 | 40 | 10 | 6 ml | 10 ml |
| | F5 | 100 | 50 | 10 | 8 ml | 10 ml |

### Characterization of Niosomes Complex

Optical microscopy was used for the characterization of the complex. The complex was suspended in a buffer, and a drop was placed on a slide and covered with a cover slip. A microscopic view of the complex was observed at a magnification of 10×10 (Junyaprasert et al., 2008).

### Entrapment Efficiency (EE)

10 mg Niosomes were added to 10 ml phosphate buffer (pH 7.4) and centrifuged at 12000 rpm for 45 min using a Remi centrifuge to separate niosomes from the unentrapped drug. The separated sedimented niosomes were treated with 0.5 ml butanol, and volume was made up to 10 ml using Acetonitrile:Water 40:60, v/v. The concentration of IRL-1620 as the supernatant was determined by using HPLC. The percentage of drug entrapment was calculated by using the formula (Singh et al., 2011),

Entrapment efficiency (%)=Weight of total drug−Weight of free drug×100 Weight of total drug

### Compatibility Studies

The compatibility between IRL-1620 and the excipients Soyalecithin and Cholesterol was evaluated using FTIR peak matching method (López-Lorente & Mizaikoff, 2016). The instrument used was Bruker FTIR (Alpha-1) over the range of 4,000 cm⁻¹ to 650 cm⁻¹.

### Particle Size Analysis

A magnetically stirred cell dispersion unit (Malvern Instruments Ltd., UK) was employed with medium-speed stirring in order to keep the niosomes dispersed during size measurement. The measurement position was 1.05 mm, and a polydisperse mode of analysis was chosen. These conditions permitted accurate measurements of particles ranging between 0.1 nm and 10000 nm [65].

### Zeta Potential Analysis

The significance of zeta potential is that its value can be related to the stability of colloidal dispersions. The values of zeta potential indicate the degree of repulsion between similarly charged niosomes in the dispersion. The zeta potential for the niosome dispersion was also determined using the Malvern instrument [66].

### Development of Topical Gel for Niosomes

The Topical Gel for Niosomes was formulated using Hydroxy Ethyl Cellulose (HEC), Triethanolamine (TEA), Polyethylene Glycol 400(PEG), Methyl Paraben, and water. Different concentrations of HEC were dispersed in water, and a suitable concentration for gel formation was determined. The idyllic ratio was found to be around 11% (w/v) solution. For the dispersion of lipophilic niosomes in the HEC gel base, TEA was used as a surfactant, and a stabilizer PEG was used. Various combinations were prepared, as shown in Table 4.

TABLE NO. 4
Composition of different IRL-1620 niosome gel formulations

| For- | | | | |
|---|---|---|---|---|
| mula- | | Surfac- | | |
| tion | Drug | tant | Stabilizer | Gelling Agent |
| G-1 | IRL-1620(1) | — | — | HEC (7.5) + Water (66) |
| G-2 | IRL-1620 (1) | TEA (2.5) | — | HEC (7.5) + Water (66) |
| G-3 | IRL-1620 (1) | TEA (2.5) | PEG (23) | HEC (7.5) + Water (66) |
| G-4 | IRL-1620 (1) | TEA (2) | PEG (23.5) | HEC (7.5) + Water (66) |
| G-5 | IRL-1620 (1) | TEA (1.5) | PEG (24) | HEC (7.5) + Water (66) |

### Details of Excipients

Hydroxyethylcellulose, a partially substituted poly(hydroxyethyl) ether of cellulose, is a nonionic, hydrophilic polymer with a linear and filiform chain, having a degree of substitution of minimum 1.5 (three hydroxyls substituted/two units) [67].

Triethanolamine is a tertiary amino compound that is ammonia in which each of the hydrogens is substituted by a 2-hydroxyethyl group. It has a role as a buffer and a surfactant. It is a tertiary amino compound, a triol, and an amino alcohol. It derives from a triethylamine. It is a conjugate base of a triethanolammonium.

PEG 400 is a low-molecular-weight grade of polyethylene glycol. It is a clear, colorless, viscous liquid. Due in part to its low toxicity, PEG 400 is widely used in a variety of pharmaceutical formulations [68].

Methylparaben is a 4-hydroxybenzoate ester resulting from the formal condensation of the carboxy group of 4-hydroxybenzoic acid with methanol. It is the most frequently used antimicrobial preservative in cosmetics. In addition, it has a role as a plant metabolite, an antimicrobial food preservative, a neuroprotective agent, and an antifungal agent [69].

### Evaluation of Niosome Gel

The Gel formulations were evaluated based on the following parameters:

Clarity: It was determined by visual inspection under the black and white background, and it was graded as turbid, clear, and very clear (glassy).

Homogeneity: It was determined by visual inspection for the appearance of the gel.

Precipitation: It was determined by visual inspection for the presence of any aggregates.

### In-Vitro Drug Release Studies

In-vitro dissolution studies for all the prepared gel were carried out through Goat Skin using Franz diffusion Cell at 100 rpm in 50 ml of phosphate buffered saline 7.4 pH as a dissolution media, maintained at 37±5° C. (Fathalla, 2014)(Helal et al., 2012). Aliquots of 0.5 ml were withdrawn at the specified time intervals of up to 48 hours, and the concentration of IRL-1620 was analyzed using HPLC. After each sampling, an equal volume of fresh media was replaced to maintain the constant volume. Then, the samples were mixed with 0.5 ml of Acetonitrile:Water (40:60, v/v), and HPLC analysis was performed.

### Stability Study

The stability of the Niosome gel formulation was evaluated by keeping in sealed glass vials and storing them in two different storage conditions, that is, refrigeration temperature (2-8° C.) and room temperature for a period of 45 days (Dantas et al., 2016). The samples were withdrawn at different time intervals over a period of study and a % assay of the gel was performed using HPLC.

### Results

### Analysis of IRL-1620 (Sovateltide)

The developed high-performance liquid chromatographic (HPLC) method for the quantification of IRL-1620 was simple and sensitive. Separation was carried out on the reversed-phase C18 column (250 mm×4.6 mm), and the column effluent was monitored by a UV detector at 215 nm. The method was found to be precise, accurate, and specific during the study. This method was successfully applied for entrapment efficiency, drug release, and stability studies.

### Characterization of Niosomes Complex

The microscopic view of the niosomes complex, at 10×10 magnification, indicated the presence of oval to spherical-shaped vesicles.

### Entrapment Efficiency (EE)

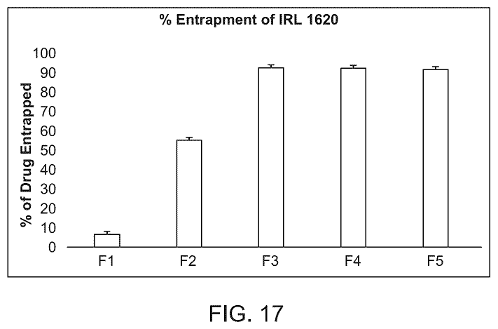

According to the drug entrapment study, the maximum drug entrapment was shown by F3. The entrapment efficiency of all formulations is graphically represented in below FIG. 17.

The niosomes prepared showed high entrapment efficiency. The formulation F3 showed the highest entrapment efficiency of 93% of IRL-1620, indicating the optimum amount of lipid required for the formation of niosomes. With further increase in the lipid concentration, the Entrapment efficiency did not change a lot, indicating that the lipid concentration did not help in entrapping the drug into the matrix.

TABLE NO. 5
Entrapment Efficiency of different formulations

| Formulation | Area | % Entrapment |
|---|---|---|
| F1 | 77023 | 6.99 |
| F2 | 612344 | 55.60 |
| F3 | 1024288 | 93.0 |
| F4 | 1022456 | 92.8 |
| F5 | 1013652 | 92.04 |

### Compatibility Studies

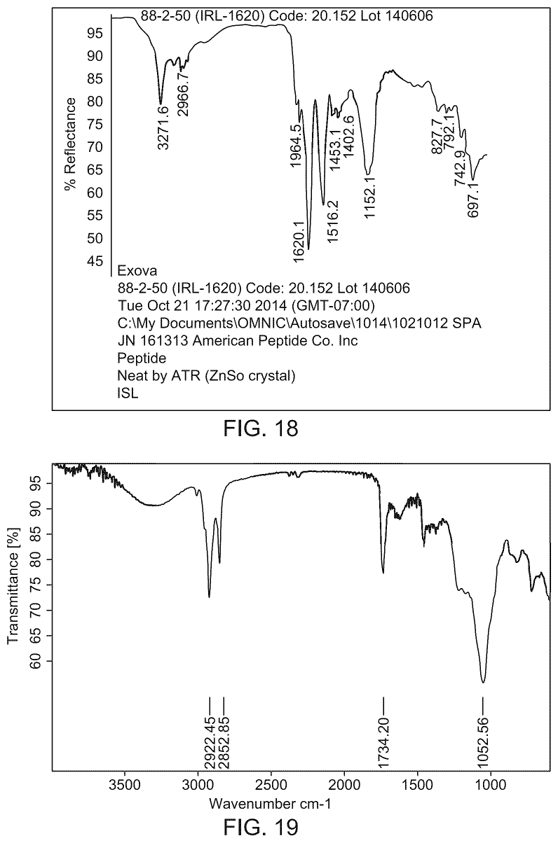

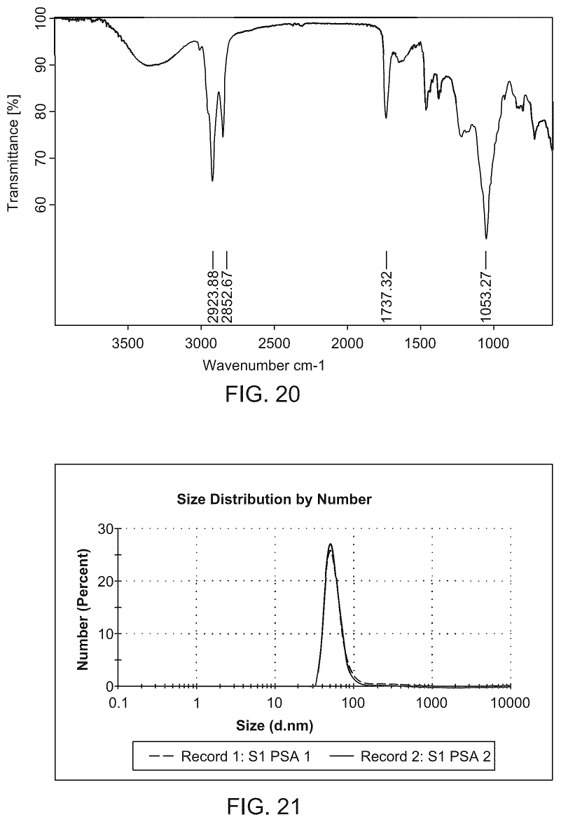

There was no appearance or disappearance of peaks of the IR spectra in the drug-lipid mixture, which confirmed the absence of any chemical interaction between the drug and lipids as shown in FIG. 18-20.

### Particle Size Analysis

Results of particle size analysis indicated that the prepared niosomes possess the particle size in the range of 218 nm (FIG. 21), and increasing the ratio of soyalecithin did not change the size of niosomes.

### Zeta Potential Analysis

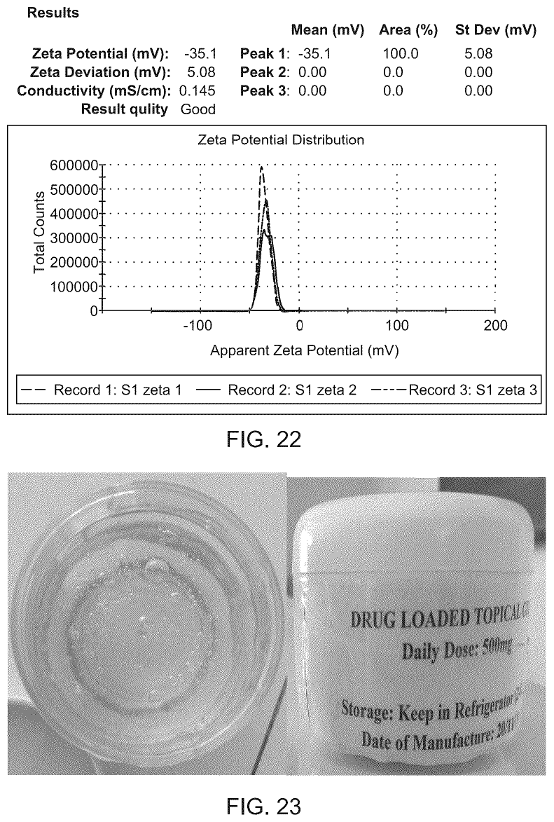

The IRL-1620 loaded niosomes had a zeta potential value of −35.1 mV, which is a measure of the net charge of the niosomes (FIG. 22). This negative charge on the surface of niosomes induces a repulsion between the particles and makes them stable and avoids agglomeration (Bayindir & Yuksel, 2010). Thus, the niosome will have acceptable stability.

### Evaluation of Niosome Gel

The Appearance, color, homogeneity, and precipitation results were observed, as depicted in Table 6. G-3 was considered to be the best combination in terms of texture, homogeneity, and spreadability.

TABLE NO. 6
Physical properties of Niosome Gels

| Formulation | Appearance | Color | Homogeneity | Precipitation |
|---|---|---|---|---|
| G-1 | Hard Gel | Clear | Homogeneous | None |
| G-2 | Gel | Clear | Homogeneous | None |
| G-3 | Gel | Clear | Homogeneous | None |
| G-4 | Gel | Turbid | Homogeneous | None |
| G-5 | Viscous liquid | Turbid | Homogeneous | None |

### In-Vitro Drug Release Studies

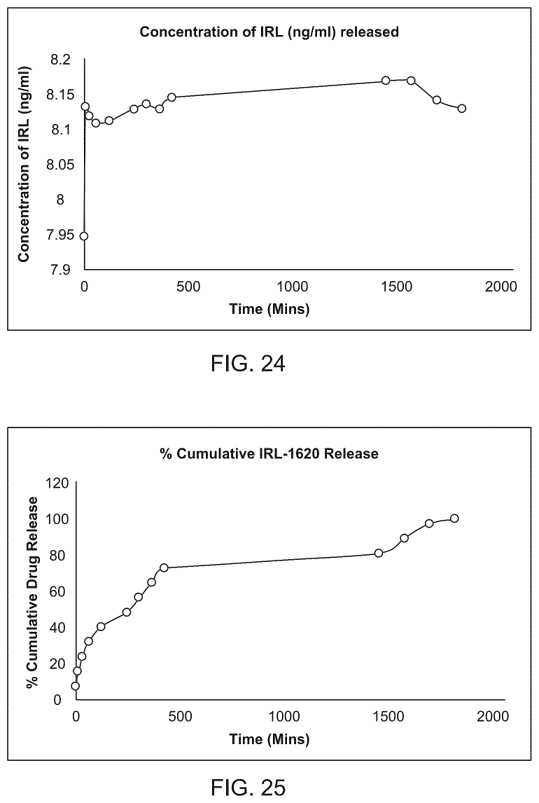

In vitro studies of niosome gel formulations (Shahiwala & Misra, 2002) were carried out in PBS pH 7.4 using Franz diffusion Cell through goat skin and results were shown in FIG. 24. It was observed from the data that in vitro drug release of IRL-1620 from niosome gel formulation was sharply increased till 6 hr followed by a maintained concentration for around 26 hours with a gradual decrease in the release. The cumulative release of IRL-1620 from the formulation was 81.1% after 24 hours (FIG. 25).

### Stability Studies

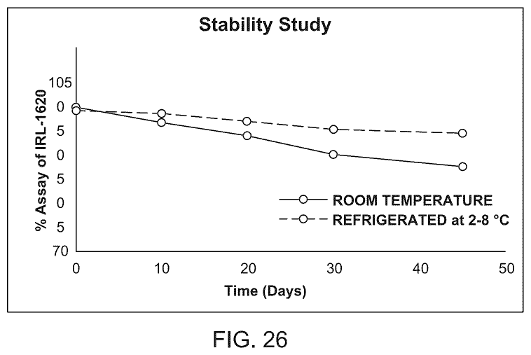

Stability studies were carried out by keeping the niosome gel formulation for one month at different storage conditions. At different time points, a sample was withdrawn, and an assay of IRL-1620 was done using the HPLC method and results are shown in FIG. 26.

It was observed from the results that there is a decrease in drug content at room temperature, whereas, in the case of refrigerated temperature, no significant change was observed. Thus, the developed niosome formulation was found to be stable at the end of the study on storage conditions.

### Conclusion

IRL-1620 niosomes entrapment efficiency ranges from 6.99% to 93.0%. The niosome formulations show particle diameters in the range of 218 nm. The compatibility studies, zeta potential, and release study data indicate that, by encapsulation of the drug into niosomes, it is possible to sustain and control the release of the drug for a longer duration with enhanced stability. The use of Hydroxy ethyl cellulose and Triethanolamine for the gel base proved to have better dispersion and release of IRL-1620 from the niosomes. Thus, the developed formulation has the ability to entrap IRL-1620, release the drug for a longer duration, and enhance the stability of the peptide moiety.

### Testing

In any of the above embodiments, testing will be needed to quantify the regeneration of damaged tissue. Visual granulation and epithelialization of the wound bed may be strategically quantified to assess wound healing. Microscopy, such as electron microscopy, may measure in vitro samples of tissue, quantifying neuroepithelium and epidermal structures. The 2-point nerve sensation testing may be employed if measuring the loss of sense. Thermal stimuli to the skin may also be used to test neural recovery. Another test to directly measure nerve conduction, such as electromyography or electroneurography, may be used.

A comparative study was performed with the two preferred embodiments with regard to acute wound healing.

### Title: Pharmacological Evaluation of Topical Wound Healing Activity of Topical Gel and Biodegradable Hydrogel Film in Excision Model and Incision Model of Wound in Rats

Study details: This study was carried out to study the effect of treatment on wound healing in rats. This study involves the excision and incision models of wound healing.

List of abbreviations: IHC: immunohistochemistry; MT: Masson's Trichome; TEM: Transmission Electron Microscopy; bFGF: Basic fibroblast growth factor; PDGF: Platelet-derived growth factor.

GLP compliance statement: This study was conducted in compliance with current principles of Good Laboratory Practices.

### Excision Model

### Objectives:

To induce excision wound and evaluate the wound healing activity of test formulations in comparison with standard drug formulation based on
wound area
epithelization time
wound contraction rate
wet granulation tissue weight
H & E, MT staining and IHC

### Material & Method:

Animals: The experimental protocol for this study was reviewed and approved by the Institutional Animal Ethics Committee, DPSRU, New Delhi in accordance with the guidelines of “The Committee for the Purpose of Control and Supervision of Experiments on Animals.” (CPCSEA, Delhi, India). Albino Wistar rats weighing 180-200 g were used in this study. Animals were acclimatized in the institutional animal house for one week prior to use. The animals were provided with free access to food and water ad libitum.

### Study Group:

| Grp | Treatment | Nomenclature | n |
|---|---|---|---|
| 1. | Disease Control | Control | 6 |
| 2. | Placebo-1 Control (placebo gel) | Placebo 1 | 3 |
| 3. | Placebo-2 Control (placebo film) | Placebo 2 | 3 |
| 4. | Positive Control (Regen-D 150) | Positive Control | 6 |
| 5. | Topical Gel OD | Treatment 1 | 6 |
| 6. | Biodegradable Hydrogel Film | Treatment 2 | 6 |
| 7. | Topical Gel OD + Standard Drug | Treatment 1 + STD | 6 |
| | (Regen-D 150) | | |
| 8. | Biodegradable Hydrogel Film + | Treatment 2 + STD | 6 |
| | Standard Drug (Regen-D 150) | | |

Animals were acclimatized for 7 days prior to the study.
Wistar albino rats were anesthetized with ketamine hydrochloride (50 mg/kg, i.p.), and their dorsal skin was shaved using hair removal cream and cleaned with 70% ethanol; an area of about ≈500 mm2 will be marked on the back of the rat by a standard ring, and then the full thickness of the marked skin was cut carefully using a scalpel.

In the placebo 1 & treatment 1 groups, the rats were applied with placebo 1 and gel topically for 16 days, respectively. Placebo 2 and biodegradable hydrogel film were applied every third day, and the film was covered with cotton, followed by gauze in placebo 2 and treatment 2 groups, respectively. In the positive control group, animals were applied with standard drug. In treatment1+std and treatment2+std groups, animals were applied with standard drug along with respective test drugs
The day of wound induction is considered the 0^{th} day.
The wound area was measured on the 4^{th}, 8^{th}, 12^{th}, and 16^{th} day and the wound contraction rate was determined.
On the 17^{th} day of the study, animals were sacrificed, and tissue was collected for H&E, IHC, and MT staining.

### Observation:

On the 5^{th} day, no epithelialization appeared on the skin.

Wound size was observed on the 4^{th}, 8^{th}, 12^{th}, and 16^{th} day (Table 7).

The day on which eschar fell off without leaving a raw wound behind was observed (Table 8)

The following parameters are observed:

TABLE 7
Wound Contraction Rate (%)

| Treatment | 4^{th} day | 8^{th} day | 12^{th} day | 16^{th} day |
|---|---|---|---|---|
| Disease control | 17.82 ± 1.12 | 38.89 ± 1.89 | 78.59 ± 1.84 | 84.24 ± 1.44 |
| Placebo 1 (placebo gel) | 17.62 ± 1.45 | 39.90 ± 1.49 | 78.35 ± 1.04 | 84.58 ± 1.14 |
| Placebo 2 (placebo film) | 17.76 ± 1.44 | 33.91 ± 1.50 | 71.37 ± 2.03 | 81.88 ± 1.42 |
| Positive control | 30.26 ± 8.77* | 69.33 ± 10.44* | 89.37 ± 3.73*** | 97.56 ± 2.89*** |
| Treatment 1 (gel) | 31.70 ± 7.85^{#} | 60.13 ± 11.12^{#} | 85.04 ± 3.99^{#} | 97.71 ± 2.45^{###} |
| Treatment 2 (film) | 24.53 ± 10.00 | 30.71 ± 9.72 | 74.07 ± 16.42 | 90.20 ± 9.36 |
| Treatment 1 (gel) + STD | 53.34 ± 10.61^{#} | 75.51 ± 13.26^{#} | 92.68 ± 8.50^{#} | 97.06 ± 7.04^{#} |
| Treatment 2(film) + STD | 38.08 ± 13.85^{@} | 62.43 ± 10.35^{@} | 90.20 ± 5.91^{@} | 96.49 ± 3.36^{@@@} |

All data are expressed as Mean ± SD,
*p ≤ 0.05,
**p ≤ 0.01,
***p ≤ 0.001 as compared with disease control;
^{#}p ≤ 0.05,
^{###}p ≤ 0.001 as compared with placebo 1;
^{@}p ≤ 0.05,
^{@@@}p ≤ 0.001 as compared with placebo 2

Statistical Analysis Report: In general, the treatment groups have improved wound contraction rate in comparison to their respective placebo groups. Positive control has significantly improved the wound contraction rate in comparison to disease control. However, treatment 2 (film) was not found to improve wound contraction rate in comparison to placebo 2 (film), but in combination with the standard drug, it has significantly produced an effect on wound contraction rate.

### 2) Epithelialization Period

TABLE 8
Epithelialization Period

| Groups | Epithelization Period (Days) |
|---|---|
| Disease Control | 15.16 ± 0.98 |
| Placebo 1 (placebo gel) | 15.00 ± 1.00 |
| Placebo 2 (placebo film) | 15.33 ± 0.57 |
| Positive control | 13.16 ± 1.32* |
| Treatment 1 (gel) | 12.33 ± 0.81^{#} |
| Treatment 2 (film) | 13.50 ± 1.64 |
| Treatment 1 (gel) + STD | 12.66 ± 1.03^{#} |
| Treatment 2 (film) + STD | 13.00 ± 1.54^{@} |

All data are expressed as Mean ± SD,
*p ≤ 0.05 as compared with disease control;
^{#}p ≤ 0.05 as compared with placebo 1;
^{@}p ≤ 0.05 as compared with placebo 2

Statistical Analysis Report: The standard drug has significantly improved the epithelialization rate compared to disease control. Treatment has significantly improved the epithelialization rate in comparison to their respective placebo. However, treatment 2 (film) was not found to improve the epithelialization period compared to placebo 2 (film); however, in combination with the standard drug, it has significantly affected the epithelialization period.

### 3) Wet Granulation Tissue Weight

The veterinary doctor has suggested that the collection of granulation tissue will hamper H&E results; therefore, wet granulation tissue weight was not assessed in this study.

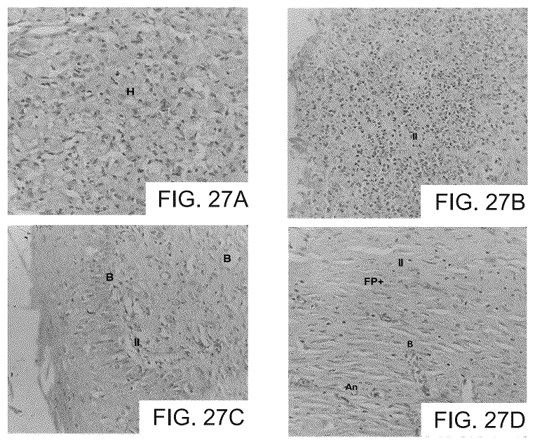

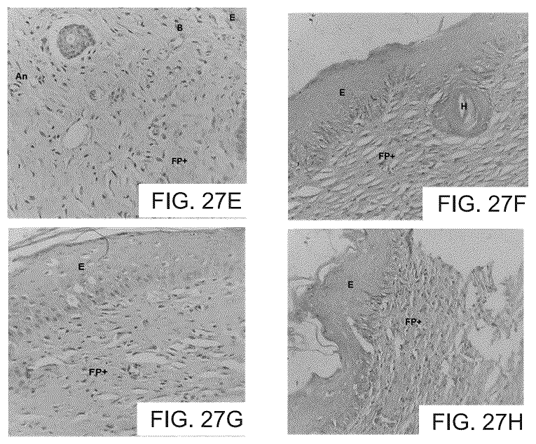

In FIG. 27: ‘II’ denotes inflammatory cell infiltration; ‘B’ denotes blood vessel; ‘An’ denotes angiogenesis; ‘FP⁺’ denotes fibroblastic proliferation; ‘E’ denotes epidermis; ‘H’ denotes histiocytes. ‘A, B, C, D, E, F, G, and H’ denotes disease control, placebo 1, placebo 2, positive control, treatment 1, treatment 2, treatment 1+std, and treatment 2+std, respectively.

### Histological Evaluation

Histological assessment of the skin was carried out by H & E staining. Neutrophil infiltration and dense inflammatory cell infiltrate (II) were observed in the placebo 1, placebo 2, and disease control groups. However, moderate inflammation was observed in treatment groups. Angiogenesis, fibroblast proliferation, and collagen re-epithelialization were not observed in placebo 1, placebo 2, and disease control groups. Different treatment has improved angiogenesis, fibroblast proliferation, and collagen deposition (FIG. 27).

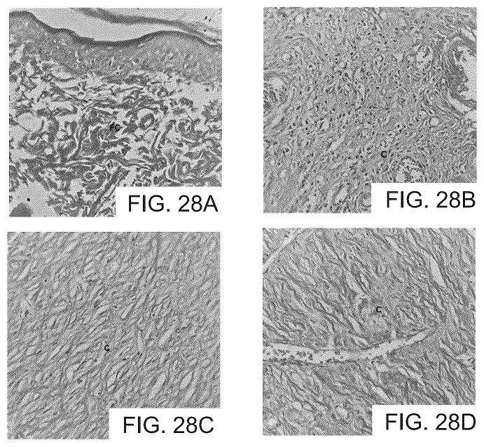

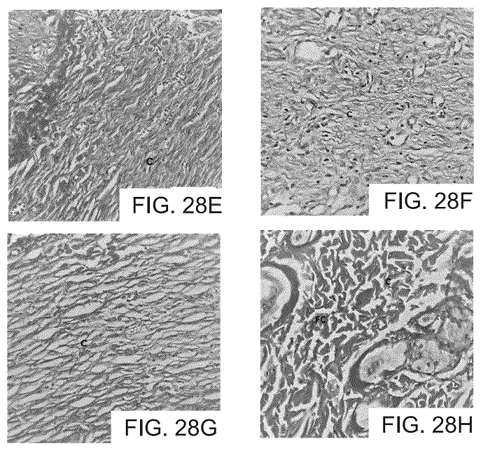

In FIG. 28: ‘C’ denotes collagen; ‘FC’ denotes fibroconnective tissue. ‘A, B, C, D, E, F, G, and H’ denotes disease control, placebo 1, placebo 2, positive control, treatment 1, treatment 2, treatment 1+std, and treatment 2+std, respectively.

MT staining: In the disease control, placebo 1 and placebo 2 groups, a mild increase in collagen deposition was observed, whereas it was found to be improved with treatment in the respective group. In the positive control, treatment 1+std and treatment 2+std groups, collagen deposition was found to be increased in the whole dermis (FIG. 28).

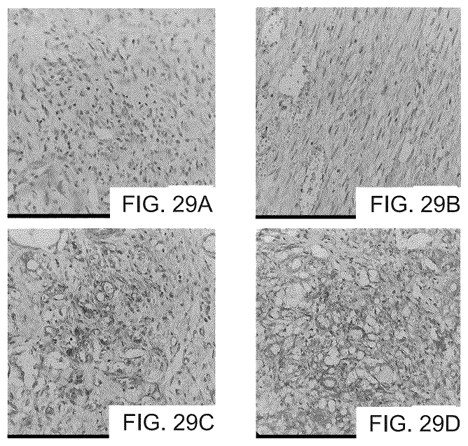

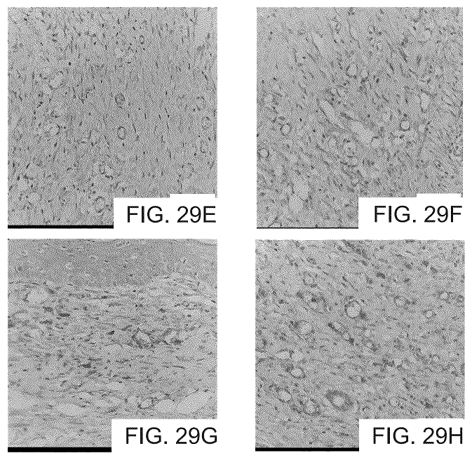

In FIG. 29: ‘A, B, C, D, E, F, G, and H’ denotes disease control, placebo 1, placebo 2, positive control, treatment 1, treatment 2, treatment 1+std, and treatment 2+std, respectively.

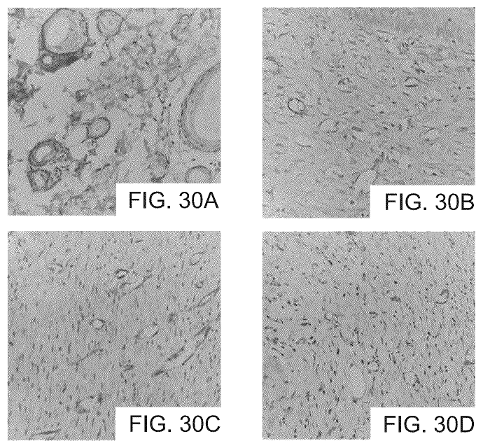

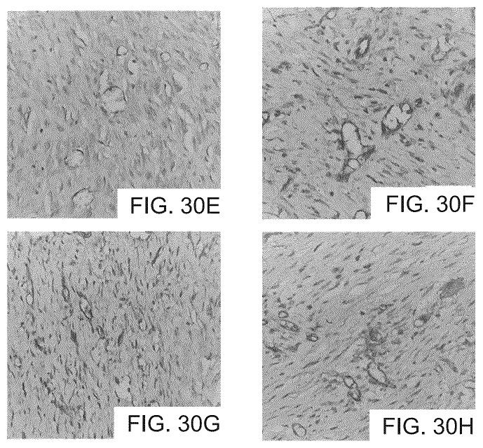

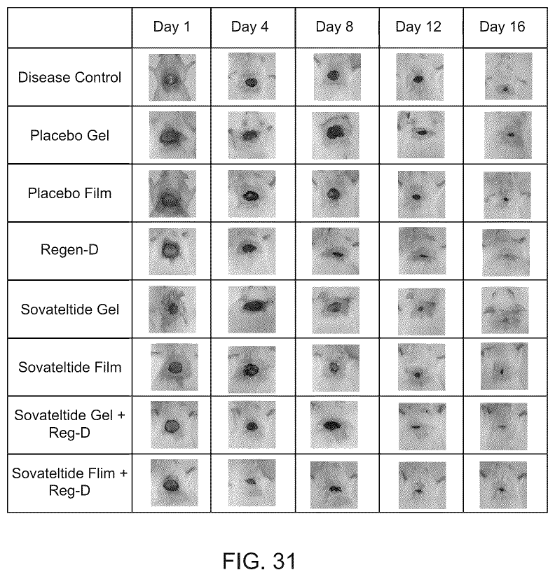

In FIG. 30: ‘A, B, C, D, E, F, G and H’ denotes disease control, placebo 1, placebo 2, positive control, treatment 1, treatment 2, treatment 1+std and treatment 2+std, respectively.

Immunohistochemistry: In placebo 1, negative expression of VEGF in the fibrotic area of the whole dermis was noted. However, in placebo 2 and disease control, weak cytoplasmic positivity of VEGF in endothelial cells of blood vessels, along with some spindle-shaped cells, was observed. In the treatment groups, a higher level of VEGF expression was observed. Moreover, fainted expression of vWF in endothelial cells of blood vessels in the fibrotic area of the whole dermis was observed in placebo 1, placebo 2, and disease control groups (FIG. 29). All the treatments have improved vWF expression; however, in treatment 2, treatment 1+std and treatment 2+std, a strong cytoplasmic positivity of vWF in endothelial cells of blood vessels was noted (FIG. 30).

Conclusion: Different treatments have improved the wound healing process by improving inflammatory parameters, fibroblast proliferation, and angiogenesis. Treatment has improved wound contraction rate and epithelialization rate. Treatments have sped up the wound healing process by improving collagen deposition and increasing VEGF and vWF expression in respective groups compared to placebo 1, placebo 2, and disease control groups.

### Incision Model

### Objectives:

To induce incision wound and application of test drugs
To measure wound breaking strength (WBS)
To collect tissue from animals for H & E, IHC, and MT staining.

### Material & Method:

Animals: The experimental protocol for the present study was reviewed and approved by the Institutional Animal Ethics Committee, DPSRU, New Delhi, in accordance with the guidelines of the “Committee for the Purpose of Control and Supervision of Experiments on Animals.” (CPCSEA, Delhi, India). Albino Wistar rats weighing 180-200 gm were used in this study. Animals were acclimatized in the institutional animal house for one week prior to using the animals for experimentation with free access to food and water ad libitum.

### Study Groups:

| Study | | |
|---|---|---|
| Groups | Treatment | n |
| 1 | Disease Control | 6 |
| 2 | Placebo-1 Control (Placebo gel) | 3 |
| 3 | Placebo-2 Control (Placebo film) | 3 |
| 4 | Positive Control (Regen-D 150) | 6 |
| 5 | Topical Gel | 6 |
| 6 | Biodegradable Hydrogel Film | 6 |
| 7 | Topical Gel + Standard Drug (Regen-D 150) | 6 |
| 8 | Biodegradable Hydrogel Film + Standard | 6 |
| | Drug (Regen-D 150) | |
| | Total | 42 |

### Method:

Animals were acclimatized for 7 days prior to the study.
Wistar albino rats were anesthetized with ketamine hydrochloride (50 mg/kg, i.p.), and their dorsal skin was shaved using an electric clipper (10 watts) and cleaned with 70% ethanol.
Two parallel 6 cm paravertebral incisions were made through the full thickness of the skin, 1 cm lateral to the midline of the vertebral column.
In placebo 2, treatment 2, and treatment 2+std groups, rats were applied with placebo film, biodegradable film, and biodegradable film+Regen D, respectively.
Wounds were closed with interrupted sutures, 1 cm apart, and with surgical sutures. The sutures were removed on the 7^{th} post wounding day.
In placebo 1, positive control, treatment 1, and treatment 1+std groups, rats were applied with a placebo gel, standard drug-Regen-D, test gel, and test gel+Regen-D, respectively, for 16 days.
The day of wound induction is considered the 0^{th} day.
On the 17^{th} day of the study, animals were sacrificed, and tissue was collected for H&E, IHC, and MT staining.

### Observation:

On the 7^{th} day, heavy pus formation was observed in animal numbers 1^{st}, 2^{nd}, 3^{rd}, and 1^{st}, 2^{nd} of placebo 1 and treatment 2, respectively.
On the 7^{th} day, heavy pus formation was observed in animal number 1^{st}, 2^{nd}, 3^{rd} of treatment 2+std
On the 10^{th} day, heavy pus formation was observed in animal number 3^{rd} of treatment 2.
Wound breaking strength was noted on the 10^{th} day of the study (Table 9).

The following parameters are observed:

TABLE 9
Wound Breaking Strength

| Groups | Wound Breaking Strength (g) |
|---|---|
| Disease Control | 181.12 ± 28.12 |
| Placebo 1 (placebo gel) | 190.97 ± 38.45 |
| Placebo 2 (placebo film) | 176.19 ± 28.47 |
| Positive control | 374.43 ± 61.004*** |
| Treatment 1 (gel) | 412.55 ± 75.06^{#} |
| Treatment 2 (film) | 391.025 ± 67.56^{@} |
| Treatment 1 (gel) + STD | 407.85 ± 71.67^{#} |
| Treatment 2 (film) + STD | 404.99 ± 29.39^{@@@} |

All data are expressed as Mean ± SD,
***p ≤ 0.001 as compared with disease control;
^{#}p ≤ 0.05 as compared with placebo 1;
^{@}p ≤ 0.05,
^{@@@}p ≤ 0.05 as compared with placebo 2

Statistical Analysis Report: Both treatments improved the wound breaking strength compared to their corresponding placebos. However, the standard group has improved wound breaking strength compared to disease control.

### Histological Evaluation (H&E)

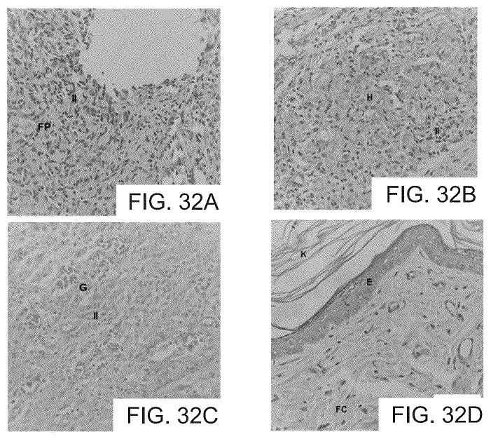

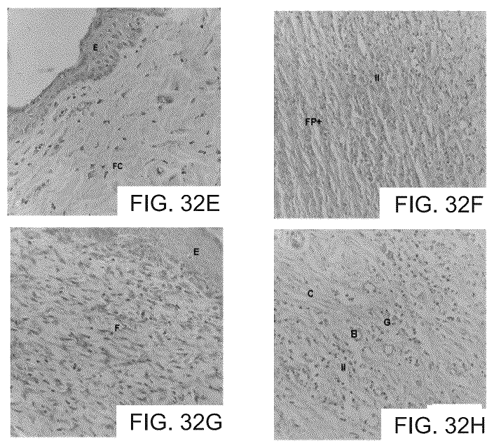

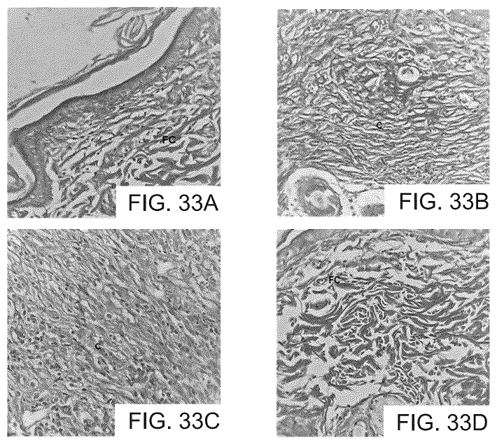

In FIG. 32: ‘II’ denotes inflammatory cell infiltration; ‘B’ denotes blood vessel; ‘An’ denotes angiogenesis; ‘FP⁺’ denotes fibroblastic proliferation; ‘E’ denotes epidermis; ‘H’ denotes hitiocytes. ‘A, B, C, D, E, F, G, and H’ denotes disease control, placebo 1, placebo 2, positive control, treatment 1, treatment 2, treatment 1+std, and treatment 2+std, respectively.

Histological evaluation: Histological evaluation was carried out by H&E staining. The placebo 1, placebo 2, and disease control groups revealed increased inflammatory parameters, whereas different treatments have reduced inflammation in comparison to placebo 1 and placebo 2 groups. However, mild inflammation was observed in the treatment 1 group. Treatment has increased the angiogenesis and fibroblast proliferation in respective groups. Moreover, a normal epidermis was observed in the treatment 1+std and treatment 2+std groups (FIG. 32).

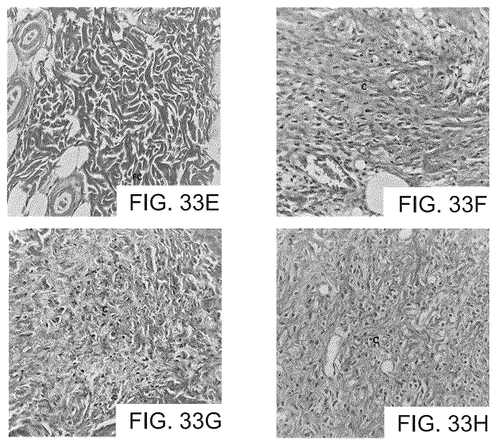

In FIG. 33: ‘C’ denotes collagen; ‘FC’ denotes fibroconnective tissue. ‘A, B, C, D. E. F. G, and H’ denotes disease control, placebo 1, placebo 2, positive control, treatment 1, treatment 2, treatment 1+std, and treatment 2+std, respectively.

MT staining: A mild increase in collagen deposition was observed in the placebo 1, 2, and disease control groups. Collagen deposition was found to be slightly higher in other groups compared to placebo 1, placebo 2, and disease control groups (FIG. 33).

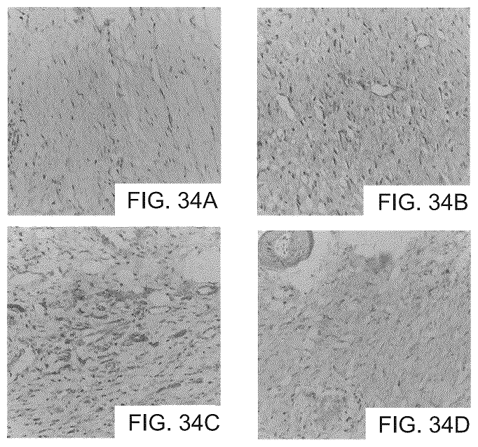

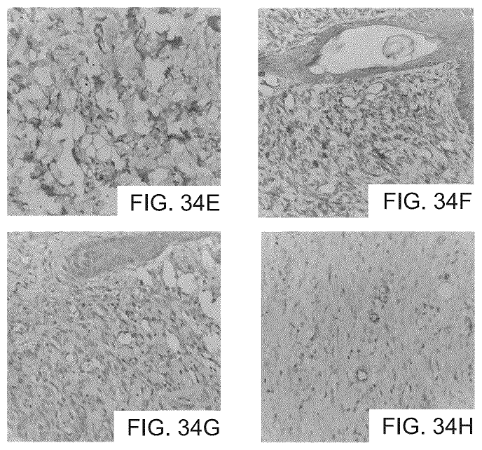

In FIG. 34 ‘A, B, C, D, E, F, G and H’ denotes disease control, placebo 1, placebo 2, positive control, treatment 1, treatment 2, treatment 1+std and treatment 2+std, respectively.

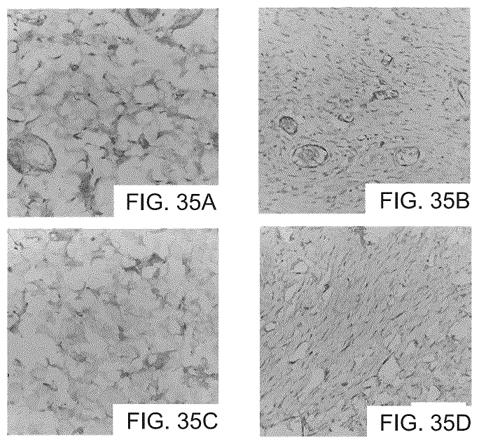

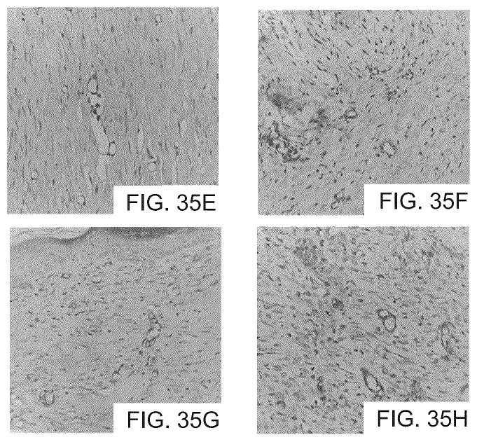

In FIG. 35: “Immunohistochemistry: to determine vWF expression

And” ‘A, B. C. D. E. F. G and H’ denotes disease control, placebo 1, placebo 2, positive control, treatment 1, treatment 2, treatment 1+std and treatment 2+std, respectively.

Immunohistochemistry: In the placebo 1, placebo 2, positive control, treatment 1, and treatment 2+std groups, a weak cytoplasmic positivity of VEGF expression in endothelial cells of blood vessels was observed while in placebo 2 and disease control groups, VEGF expression in endothelial cells of blood vessels was found to be absent. In the treatment 2 and treatment 1+std groups, an increased expression of VEGF was noted (FIG. 8). In placebo 2 and disease control groups, expression of vWF was found to be absent. In the placebo 1, positive control, treatment 1, treatment 2, and treatment 1+std groups, a weak cytoplasmic positivity of vWF in endothelial cells of blood vessels was noted, while in the treatment 1+std group, a higher expression of vWF was observed in comparison to the other groups (FIG. 35).

Conclusion: Treatments have improved fibroblast proliferation, collagen deposition, and angiogenesis. Treatments have slightly improved levels of VEGF and vWF in respective groups. Treatments have significantly improved the different parameters in comparison to the placebo group. Moreover, the standard group has significantly increased WBS compared to the normal control.

What is claimed is:

1. A method for treating dermal or cutaneous wounds and damaged cranial or peripheral nerves using a compound that includes endothelin analogs by the mechanism of anti-apoptotic, anti-inflammatory, and anti-oxidant activity that limits the death of cells and promotes the formation of new neurons (neurogenesis) and new blood vessels (angiogenesis) enhancing the healing of wounds and regenerating nerves.

2. The method of claim 1, wherein endothelin B receptor analogs are selected from the group consisting of N-Succinyl-[Glu⁹, Ala^{11,15}] endothelin 1 (sovateltide, IRL-1620), BQ-3020, [Ala^{1,3,11,15}]-endothelin, sarafotoxin S6c, and endothelin 3.

3. A composition comprising (a) an endothelin-B (ETB) receptor agonist, (b) growth factors, or osmotic agents such as glycerol, or diuretics such as furosemide or ethacrynic acid, or a steroid, or transplanted stem cells, or ferulic acid, or arctigenin, or espin isoform 1 polypeptide, or neurotrophin selected from the group consisting of glial cell line-derived neurotrophic factor, brain-derived neurotrophic factor, neurotrophin-3, neurotrophin-4/5, and ciliary neurotrophic factor, or human insulin-like growth factor 1, or Netrin1, or a mixture thereof, and optionally (c) an excipient.

4. An article of manufacture comprising:
(a) a packaged composition comprising an endothelin-B (ETB) receptor agonist and steroid or a salt thereof;
(b) an insert providing instructions for a simultaneous or sequential administration of the ETB receptor agonist and the steroid or salt thereof to treat a patient; and
(c) a container for (a) and (b).

5. The article of claim 4, wherein the endothelin-B receptor agonist is N-Succinyl-[Glu⁹, Ala^{11,15}] endothelin 1 (sovateltide, IRL-1620).

6. The method of claim 2, wherein endothelin B receptor analogs are delivered topically, sub-dermally, subcutaneously, intramuscularly, intravenously, or orally.

7. The method of claim 2, wherein endothelin B receptor analogs as such or conjugated with nanoparticles including stealth nanoparticles, hydrogels made into various formulations such as gels, creams, solutions, lotions, ointments, adhesive patches, and sprays.

8. The method of claim 2, wherein endothelin B receptor analogs are administered via a sustained release drug delivery system such as Gelfoam®, Hyaluronic gel, Seprapack™, Thiol-modified Hyaluronic acid, Glutaraldehyde cross-linking of porcine type-collagen, gelatin, chitosan glycosylated derivative, Poloxamer 407, chitosan glycerophosphate hydrogel, sustained release is Lipid core nanocapsules poly L-lysine (HBPL) nanoparticles.

9. The method of claim 1, wherein endothelin analogs, specifically endothelin B receptor analogs, are used for the treatment of wounds and or promoting wound healing and for mammalian nerve regeneration of a damaged nerve.

10. The method of claim 1, wherein endothelin analogs administration may be combined with growth factors, or osmotic agent such as glycerol, or diuretics such as furosemide or ethacrynic acid, or a steroid, or transplanted stem cells, or ferulic acid, or arctigenin, or espin isoform 1 polypeptide, or neurotrophin selected from the group consisting of glial cell line-derived neurotrophic factor, brain-derived neurotrophic factor, neurotrophin-3, neurotrophin-4/5, and ciliary neurotrophic factor, or human insulin-like growth factor 1, or Netrin1, or a mixture thereof.

11. The method of claim 1, wherein endothelin analog dose range is 0.00001 to about 1 mg and may be administered once or multiple times in a day, weeks, or months.
